(19) United States
(12) Patent Application Publication
(10) Pub. No.: US 2023/0335286 A1
(43) Pub. Date: Oct. 19, 2023

(54) SUPERVISORY DATA GENERATION APPARATUS

(71) Applicant: NEC Corporation, Minato-ku, Tokyo (JP)

(72) Inventors: Yuji OHNO, Tokyo (JP); Toshinori HOSOl, Tokyo (JP); Masahiro KUBO, Tokyo (JP)

(73) Assignee: NEC Corporation, Minato-ku, Tokyo (JP)

(21) Appl. No.: 18/025,460

(22) PCT Filed: Sep. 17, 2020
(86) PCT No.: PCT/JP2020/035294

§ 371 Date: Mar. 9, 2023

(51) Int. Cl. G16H 50/30; A61B 5/11; A61B 5/16; G06N 20/00

(52) U.S. Cl. CPC G16H 50/30; A61B 5/11; A61B 5/165; G06N 20/00

(57) ABSTRACT

A supervisory data generation apparatus 600 includes an acquisition unit 621 configured to acquire body motion data according to a motion of a body of a target and vital data of the target, a calculation unit 622 configured to calculate a body motion score serving as an index for determining an agitation state based on the body motion data acquired by the acquisition unit 621, a determination unit 623 configured to determine whether the target is agitated using the body motion score, and a labeling unit 624 configured to label the vital data based on a result of the determination made by the determination unit 623.

### TECHNICAL FIELD

The present invention relates to a supervisory data generation apparatus, a supervisory data generation method, a training apparatus, a training method, an agitation determination apparatus, a retraining method, and a recording medium.

### BACKGROUND ART

When a patient is in an agitated state, a risk of extubation, needle dislodgement, decannulation, tumble, a fall, and the like increases, and the patient may be, for example, injured as a result thereof. In this regard, there have been known techniques for determining a predictive sign of agitation to reduce such a risk and a possibility.

Examples of known documents discussing the techniques for determining a predictive sign of agitation include Patent Literature 1. Patent Literature 1 discusses a biological information processing system including a determination unit and an estimation unit. According to Patent Literature 1, the determination unit determines discrimination information indicating whether or not a condition of a patient has changed in comparison with a normal state based on features of biological information of the patient. Then, the estimation unit estimates countermeasure information for the patient based on the discrimination information determined by the determination unit and countermeasure prediction parameters learned in advance. Further, Patent Literature 1 discloses a heartbeat and the like as one example of the biological information, and discloses an agitation score indicating a possibility that the patient is in an agitated state as one example of the discrimination information.

### CITATION LIST

### Patent Literature

Patent Literature 1: International Publication No. 2019/073927

### SUMMARY

### Technical Problem

In the case of the technique discussed in Patent Literature 1, the agitation score is calculated using a model generated as a result of carrying out machine learning in advance. Therefore, in the case of the technique discussed in Patent Literature 1, an attempt to calculate an agitation score adapted to a new hospital or a new clinical department raises a necessity of generating supervisory data for carrying out machine learning to, for example, newly train a model or retrain an existing model. However, the generation of the supervisory data takes a lot of time and effort, as it requires, for example, manual labeling based on video data.

Under these circumstances, one of objects of the present invention is to provide a supervisory data generation apparatus, a supervisory data generation method, a training apparatus, a training method, an agitation determination apparatus, a retraining method, and a recording medium capable of allowing supervisory data to be efficiently generated.

### Solution to Problem

To achieve the above-described object, according to one aspect of the present disclosure a supervisory data generation apparatus is configured to include
an acquisition unit configured to acquire body motion data according to a motion of a body of a target and vital data of the target,
a calculation unit configured to calculate a body motion score serving as an index for determining an agitation state based on the body motion data acquired by the acquisition unit,
a determination unit configured to determine whether the target is agitated using the body motion score, and
a labeling unit configured to label the vital data based on a result of the determination made by the determination unit.

Further, according to another aspect of the present disclosure, a supervisory data generation method is configured to cause a computer to
acquire body motion data according to a motion of a body of a target and vital data of the target,
calculate a body motion score serving as an index for determining an agitation state based on the acquired body motion data,
determine whether the target is agitated based on the body motion score, and
label the vital data based on a result of the determination.

Further, according to another aspect of the present disclosure, a recording medium records a program for causing a computer to realize processing including
acquiring body motion data according to a motion of a body of a target and vital data of the target,
calculating a body motion score serving as an index for determining an agitation state based on the acquired body motion data,
determining whether the target is agitated based on the body motion score, and
labeling the vital data based on a result of the determining.

Further, according to another aspect of the present disclosure, a training apparatus is configured to include
an acquisition unit configured to acquire body motion data according to a motion of a body of a target and vital data of the target,
a calculation unit configured to calculate a body motion score serving as an index for determining an agitation state based on the body motion data acquired by the acquisition unit,
a determination unit configured to determine whether the target is agitated based on the body motion score,
a labeling unit configured to label the vital data based on a result of the determination made by the determination unit, and
a training unit configured to generate a vital model configured to output a vital score serving as an index for determining the agitation state by carrying out training using the labeled vital data as supervisory data.

Further, according to another aspect of the present disclosure, a training method is configured to cause a computer to
acquire body motion data according to a motion of a body of a target and vital data of the target,
calculate a body motion score serving as an index for determining an agitation state based on the acquired body motion data,
determine whether the target is agitated based on the body motion score,
label the vital data based on a result of the determination, and
generate a vital model configured to output a vital score serving as an index for determining the agitation state by carrying out training using the labeled vital data as supervisory data.

Further, according to another aspect of the present disclosure, a recording medium records a program for causing a computer to realize processing including
acquiring body motion data according to a motion of a body of a target and vital data of the target,
calculating a body motion score serving as an index for determining an agitation state based on the acquired body motion data,
determining whether the target is agitated based on the body motion score,
labeling the vital data based on a result of the determining, and
generating a vital model configured to output a vital score serving as an index for determining the agitation state by carrying out training using the labeled vital data as supervisory data.

Further, according to another aspect of the present disclosure, an agitation determination apparatus is configured to include
an acquisition unit configured to acquire body motion data according to a motion of a body of a target and vital data according to a state of the body of the target,
a calculation unit configured to calculate a body motion score serving as an index for determining an agitation state based on the body motion data acquired by the acquisition unit and also calculate a vital score serving as an index for determining the agitation state based on the vital data acquired by the acquisition unit,
a determination unit configured to determine to retrain a vital model used when the vital score is calculated based on the body motion score and the vital score calculated by the calculation unit, and
a retraining unit configured to retrain the vital model according to a result of the determination unit.

Further, according to another aspect of the present disclosure, a retraining method is configured to cause a computer to
acquire body motion data according to a motion of a body of a target and vital data according to a state of the body of the target,
calculate a body motion score serving as an index for determining an agitation state based on the acquired body motion data and also calculate a vital score serving as an index for
determining the agitation state based on the acquired vital data, determine to retrain a vital model used when the vital score is calculated based on the calculated body motion score and vital score, and
retrain the vital model according to a result of the determination.

Further, according to another aspect of the present disclosure, a recording medium records a program for causing a computer to realize processing including
acquiring body motion data according to a motion of a body of a target and vital data according to a state of the body of the target,
calculating a body motion score serving as an index for determining an agitation state based on the acquired body motion data and also calculating a vital score serving as an index for determining the agitation state based on the acquired vital data,
determining to retrain a vital model used when the vital score is calculated based on the calculated body motion score and vital score, and
retraining the vital model according to a result of the determining.

### Advantageous Effects of Invention

According to each of the above-described configurations, the present invention can provide a supervisory data generation apparatus, a supervisory data generation method, a training apparatus, a training method, an agitation determination apparatus, a retraining method, and a recording medium capable of allowing supervisory data to be efficiently generated.

### BRIEF DESCRIPTION OF DRAWINGS

FIG. 1 is a block diagram illustrating an example of the configuration of a model training apparatus according to a first exemplary embodiment of the present disclosure.

FIG. 2 is a diagram illustrating one example of information stored in a storage unit.

FIG. 3 is a diagram illustrating one example of body motion data included in sensing data illustrated in FIG. 2.

FIG. 4 is a diagram illustrating one example of vital data included in the sensing data illustrated in FIG. 2.

FIG. 5 is a diagram illustrating one example of a body motion score included in body motion score information illustrated in FIG. 2.

FIG. 6 is a diagram illustrating an example of processing by an agitation determination unit.

FIG. 7 is a flowchart illustrating examples of operations of the model training apparatus according to the first exemplary embodiment of the present disclosure.

FIG. 8 is a flowchart illustrating one example of the agitation determination processing illustrated in FIG. 6.

FIG. 9 is a block diagram illustrating another example of the configuration of the model training apparatus.

FIG. 10 is a diagram illustrating an example of the configuration of an agitation determination system according to a second exemplary embodiment of the present disclosure.

FIG. 11 is a block diagram illustrating an example of the configuration of a sensor apparatus illustrated in FIG. 9.

FIG. 12 is a block diagram illustrating an example of the configuration of a bed terminal illustrated in FIG. 9.

FIG. 13 is a block diagram illustrating an example of the configuration of an agitation determination apparatus illustrated in FIG. 9.

FIG. 14 is a diagram illustrating one example of information stored in a storage unit.

FIG. 15 is a diagram illustrating one example of a vital score included in vital score information illustrated in FIG. 14.

FIG. 16 is a diagram for illustrating an example of processing by an agitation state determination unit.

FIG. 17 is a diagram for illustrating an example of processing by the agitation state determination unit.

FIG. 18 is a flowchart illustrating examples of operations of the agitation determination apparatus.

FIG. 19 is a block diagram illustrating another example of the configuration of the agitation determination apparatus.

FIG. 20 is a diagram illustrating one example of another information stored in the storage unit.

FIG. 21 is a diagram illustrating the hardware configuration of a supervisory data generation apparatus according to a third exemplary embodiment of the present disclosure.

FIG. 22 is a block diagram illustrating an example of the configuration of the supervisory data generation apparatus.

### DESCRIPTION OF EMBODIMENTS

### First Exemplary Embodiment

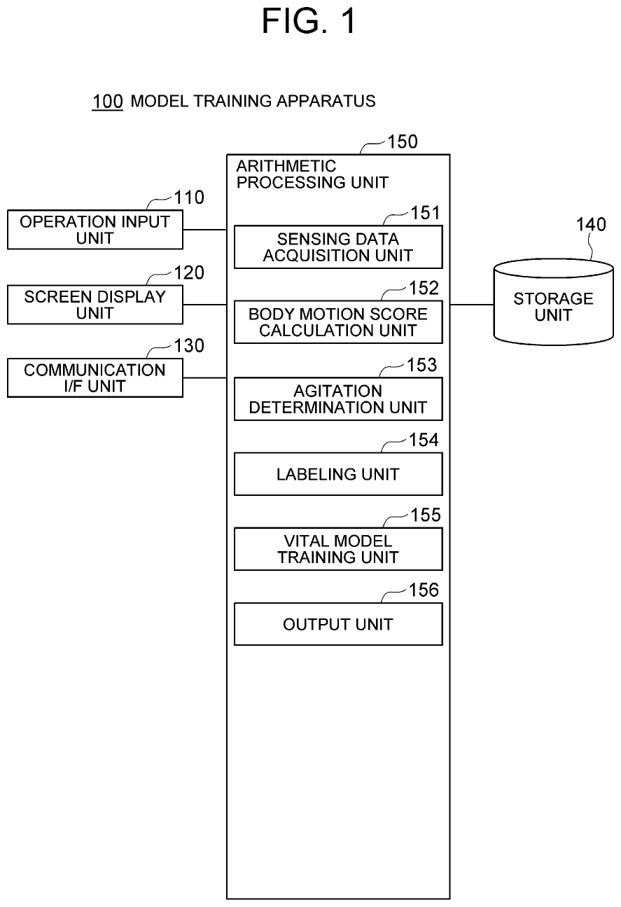

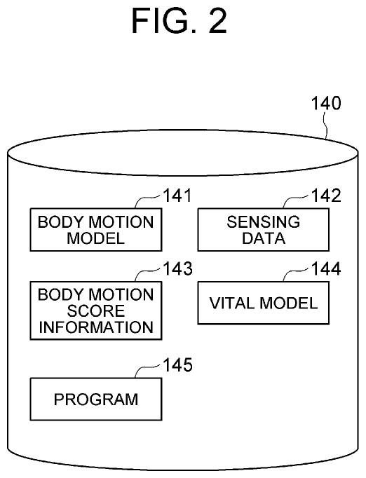

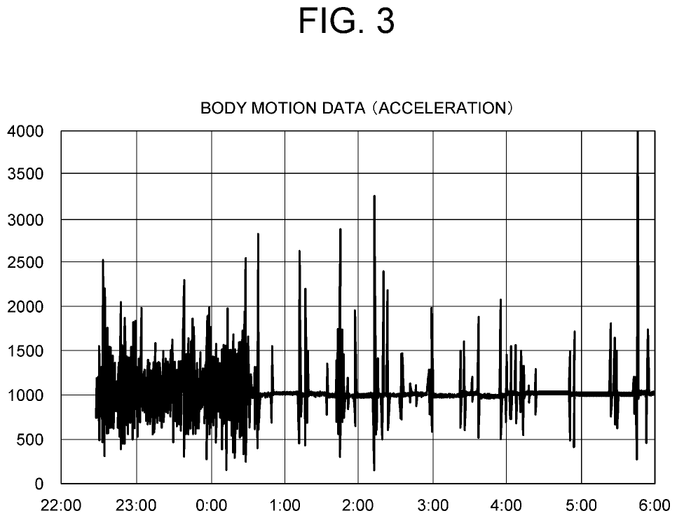

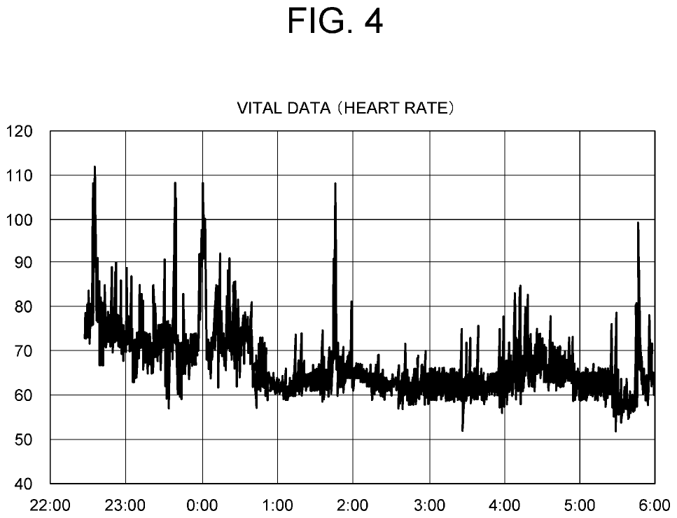

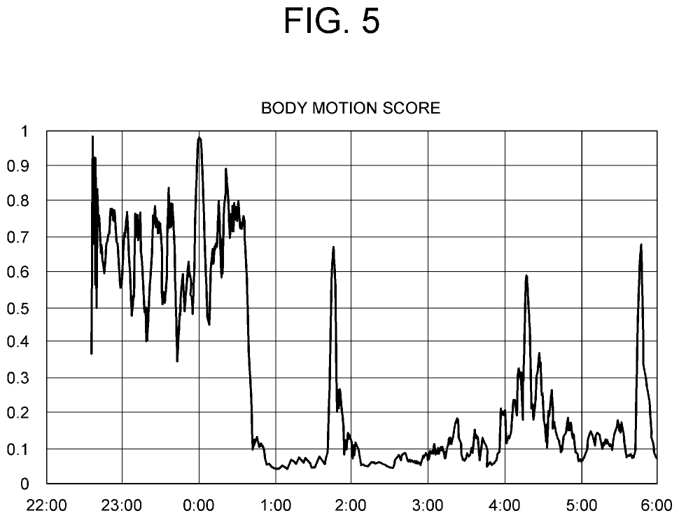

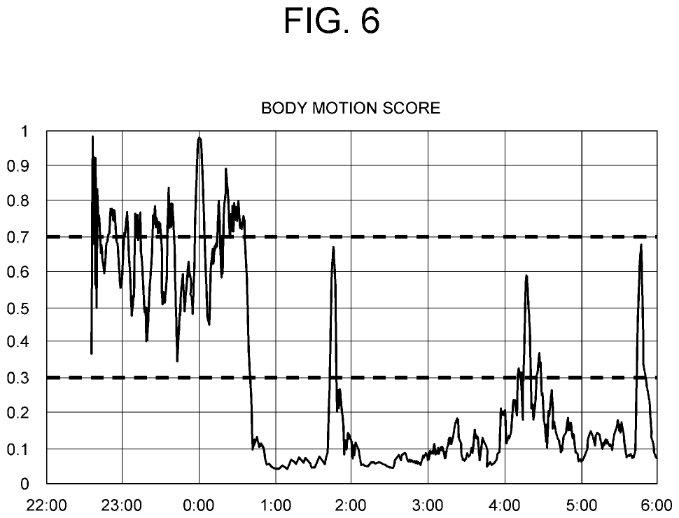

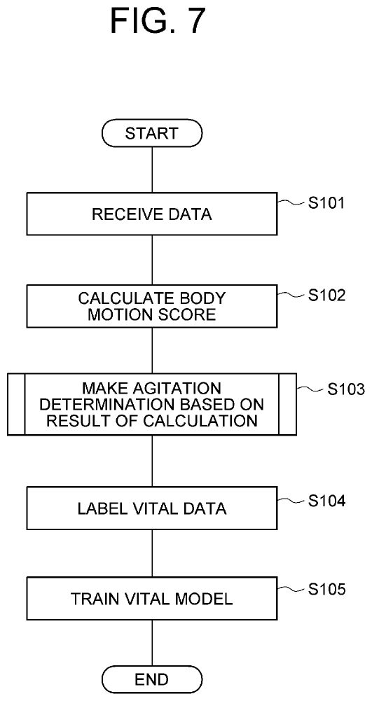

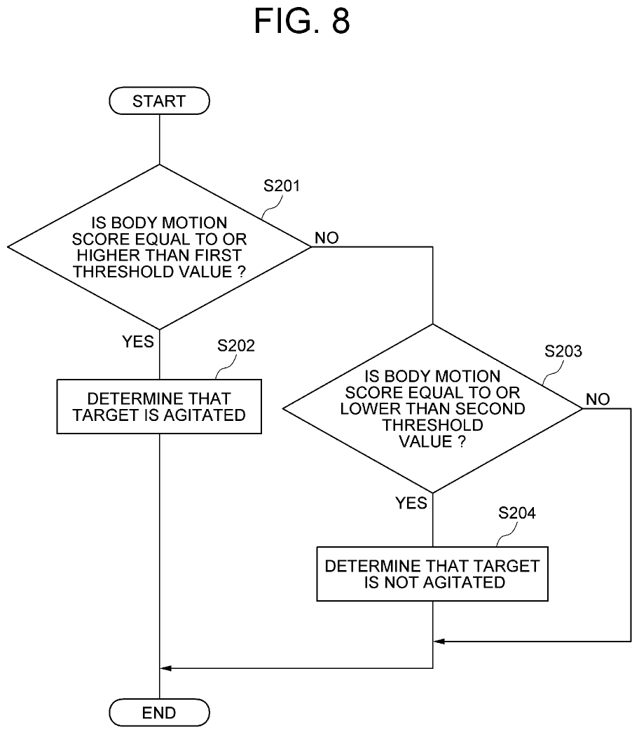

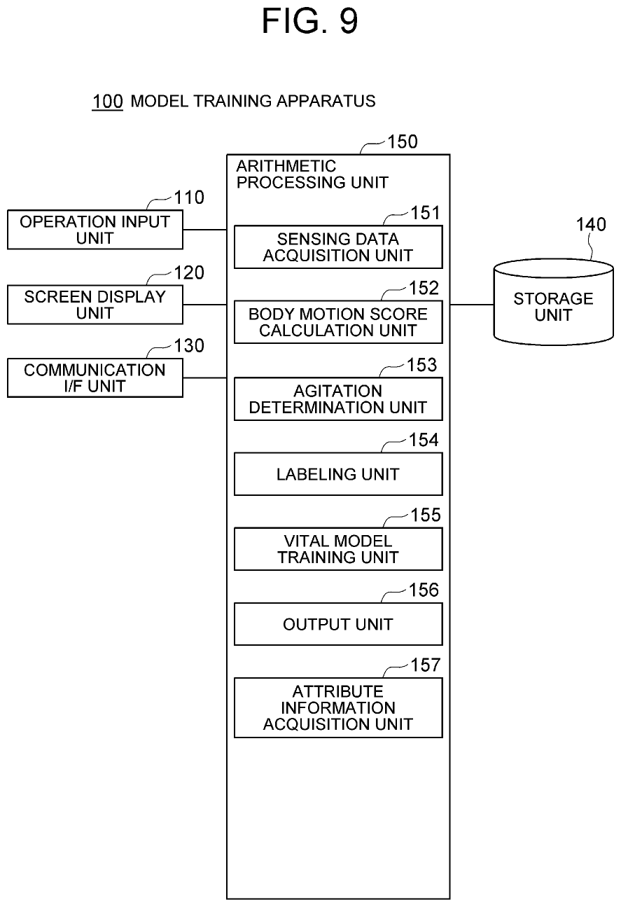

A first exemplary embodiment of the present disclosure will be described with reference to FIGS. 1 to 9. FIG. 1 is a block diagram illustrating an example of the configuration of a model training apparatus 100. FIG. 2 is a diagram illustrating one example of information stored in a storage unit 140. FIG. 3 is a diagram illustrating one example of body motion data included in sensing data 142. FIG. 4 is a diagram illustrating one example of vital data included in the sensing data 142. FIG. 5 is a diagram illustrating one example of a body motion score included in body motion score information 143. FIG. 6 is a diagram illustrating an example of processing by an agitation determination unit 153. FIG. 7 is a flowchart illustrating examples of operations of the model training apparatus 100. FIG. 8 is a flowchart illustrating one example of agitation determination processing. FIG. 9 is a block diagram illustrating another example of the configuration of the model training apparatus 100.

The first exemplary embodiment of the present disclosure will be described regarding a model training apparatus 100, which trains a vital model 144. As will be described below, the model training apparatus 100 makes an agitation determination using a body motion score calculated based on body motion data acquired by a sensing data acquisition unit 151. Further, the model training apparatus 100 generates supervisory data by labeling vital data acquired by the sensing data acquisition unit 151 based on the result of the agitation determination. Then, the model training apparatus 100 generates the vital model 144 by carrying out training using the generated supervisory data. In other words, the model training apparatus 100 creates the vital model 144 based on the acquired vital data and body motion data.

Note that the vital model 144 generated by the model training apparatus 100, which will be described in the present exemplary embodiment, can be used when an agitation state of a target such as a patient is determined in a scene such as an acute hospital, a convalescent hospital, a care facility, or monitoring at home. The vital model 144 generated by the model training apparatus 100 may be used when the agitation state is determined in a scene different from the above-described examples.

In the present exemplary embodiment, the agitation refers to a state in which the patient is restless and upset. The agitation may be caused by delirium or the like. Further, the agitation state indicates a state regarding the agitation of the patient. The agitation state indicates, for example, whether the patient is agitated or whether the patient exhibits a predictive sign of agitation. Note that the agitation state may include another index regarding a likelihood of the patient's agitation. When the patient is agitated, the patient may conduct a problematic behavior, such as falling off the bed, removing intubation, shouting abnormally, or committing violence. Therefore, it is desirable to accurately determine the agitation state.

The model training apparatus 100 is an information processing apparatus that generates the vital model 144 by carrying out the training using the supervisory data generated based on the body motion data and the vital data. For example, the model training apparatus 100 generates the vital model 144 by carrying out machine learning using a support vector machine (SVM), a neural network, or the like. FIG. 1 illustrates an example of the configuration of the model training apparatus 100. Referring to FIG. 1, the model training apparatus 100 includes, for example, an operation input unit 110, a screen display unit 120, a communication I/F unit 130, the storage unit 140, and an arithmetic processing unit 150 as main constituent elements thereof. The model training apparatus 100 is, for example, an information processing apparatus such as a personal computer or a tablet used by medical staff such as a doctor or a nurse, a server set up in a hospital or the like, or a cloud server. The model training apparatus 100 may be a combination of the information processing apparatus such as a personal computer or a tablet, and the server and/or the like.

The operation input unit 110 is configured of an operation input device such as a keyboard and/or a mouse. The operation input unit 110 detects an operation input by an operator or the like operating the model training apparatus 100, and outputs it to the arithmetic processing unit 150.

The screen display unit 120 is configured of a screen display device such as an LCD (a Liquid Crystal Display). The screen display unit 120 can display various kinds of information stored in the storage unit 140, such as the sensing data 142 and the body motion score information 143, on the screen according to an instruction from the arithmetic processing unit 150.

The communication IF unit 130 is configured of a data communication circuit. The communication IF unit 130 carries out data communication between the model training apparatus 100 and an external apparatus or the like to which the model training apparatus 100 is connected.

The storage unit 140 is a storage device such as a hard disk or a memory. FIG. 2 illustrates one example of information stored in the storage unit 140. As illustrated in FIG. 2, the storage unit 140 stores processing information required for various kinds of processing by the arithmetic processing unit 150, and a program 145 therein. The program 145 realizes the various kinds of processing units by being read in and executed by the arithmetic processing unit 150. The program 145 is read in from an external apparatus or a recording medium in advance via a data input/output function such as the communication I/F unit 130, and is stored in the storage unit 140. Examples of main information stored in the storage unit 140 include a body motion model 141, the sensing data 142, the body motion score information 143, and the vital model 144.

The body motion model 141 is a model for calculating a body motion score based on the body motion data acquired by the sensing data acquisition 151. For example, the body motion model 141 receives information according to the body motion data as an input, and outputs the body motion score. The body motion model 141 is, for example, a trained model generated in advance by carrying out machine learning using a support vector machine (SVM), a neural network, or the like in an external apparatus or the like. For example, the machine learning is carried out by using data generated by labeling previously measured body motion data with the presence or absence of the agitation as supervisory data. The body motion model 141 is acquired from the external apparatus or the like via the communication I/F unit 130 or the like, and is stored in the storage unit 140.

Note that the body motion score is an index for determining whether the target such as the patient is agitated. Generally, the body motion score is an index indicating the patient's motion itself, and therefore is environment-independent. The body motion score is, for example, a value from 0 to 1, inclusive. The body motion score indicates a higher likelihood that the target is agitated as the value thereof is closer to 1, and indicates a higher likelihood that the target is not agitated as the value thereof is closer to 0. The body motion score may be an index expressed using two values that are 1 indicating that the target is agitated and 0 indicating that the target is not agitated. The body motion score may be an index indicating the intensity of the agitation, such as an index set to 2 for strong agitation and 1 for slight agitation.

Further, the data input to the body motion model 141 may be time-series data itself of the body motion data, or may be various kinds of feature amounts calculated by subjecting the time-series data to processing for converting the time-series data into the feature amount such as averaging processing or differential processing. Further, the body motion data may include a plurality of types of data, as will be described below. The body motion model 141 may be configured to input only one type of body motion data or may be configured to input a plurality of types of body motion data. Further, the body motion model 141 may include only one type of model corresponding to one type of body motion data or may include a plurality of types of models according to various types of body motion data.

The sensing data 142 includes the time-series body motion data acquired by the sensing data acquisition unit 151 and time-series vital data at times corresponding to the body motion data.

Now, the body motion data included in the sensing data 142 refers to a physical amount regarding the motion of the patient's body. For example, the body motion data includes at least one of an acceleration, an angular velocity, or an angle at a predetermined portion such as the patient's arm, body, or foot, a vocal volume, or the like. The body motion data can be acquired using a sensor such as an acceleration sensor, a gyro sensor (an angular velocity sensor), an angular sensor, or a microphone worn by the target. For example, FIG. 3 illustrates one example of the time-series data of the acceleration. In the case of FIG. 3, an x axis represents time and a y axis represents the magnitude of the acceleration.

Note that the sensing data 142 may include only one type of body motion data such as the acceleration or may include a plurality of types of body motion data such as the acceleration and the vocal volume.

Further, the vital data included in the sensing data 142 refers to a physical amount varying according to the vital activity of the patient. For example, the vital data includes at least one of a heart rate, a respiratory rate, a blood pressure value, a body temperature, a skin temperature, a blood flow rate, a blood oxygen saturation level, or the like of the patient. The vital data can be acquired using a sensor such as a heart rate sensor, a respiratory rate sensor, a blood pressure sensor, a body temperature sensor, or a blood oxygen saturation level sensor worn by the target. For example, FIG. 4 illustrates one example of the time-series data of the heart rate. In the case of FIG. 4, an x axis represents time and a y axis represents the heart rate.

Note that the sensing data 142 may include only one type of vital data such as the heart rate or may include a plurality of types of vital data such as the heart rate and the blood pressure value, similarly to the example in the case of the body motion data.

The body motion score information 143 includes the body motion score, which is the index for determining whether the target is agitated. A labeling unit 154, which will be described below, carries out labeling based on the body motion score. For example, the body motion score information 143 includes the body motion score at each time.

FIG. 5 illustrates one example of the body motion score calculated by the body motion score calculation unit 152 based on the body motion data illustrated in FIG. 3. In the case of FIG. 5, an x axis represents time and a y axis represents the body motion score. As illustrated in FIG. 5, the body motion score is expressed by, for example, a value from 0 to 1, inclusive. The body motion score indicates a higher likelihood that the target is agitated as the value thereof is closer to 1, and indicates a higher likelihood that the target is not agitated as the value thereof is closer to 0.

The vital model 144 (a state model) is a trained model generated by a vital model training unit 155. For example, the vital model 144 receives information according to the vital data as an input, and outputs the vital score, which is a state score. The details of processing in which the vital model training unit 155 generates the vital model will be described below.

The arithmetic processing unit 150 includes a microprocessor such as an MPU and a peripheral circuit thereof. The arithmetic processing unit 150 reads in the program 145 from the storage unit 140 and executes it, thereby causing the above-described hardware and the program 145 to cooperate with each other to realize various kinds of processing units. Examples of main processing units realized by the arithmetic processing unit 150 include the sensing data acquisition unit 151, the body motion score calculation unit 152, the agitation determination unit 153, the labeling unit 154, the vital model training unit 155, and an output unit 156.

The sensing data acquisition unit 151 acquires the time-series body motion data and the time-series vital data from an external apparatus such as a sensor apparatus via the communication IF unit 130. For example, the sensing data acquisition unit 151 acquires the body motion data and the vital data acquired at the same time as each other from the sensor apparatus. Then, the sensing data acquisition unit 151 stores the acquired body motion data and vital data into the storage unit 140 as the sensing data 142.

The body motion score calculation unit 152 calculates the body motion score using the body motion model 141.

For example, the body motion score calculation unit 152 acquires the time-series body motion data like the example illustrated in FIG. 3 by referring to the sensing data 142. Further, the body motion score calculation unit 152 inputs the acquired data to the body motion model 141 to calculate the time-series body motion score like the example illustrated in FIG. 5. After that, the body motion score calculation unit 152 stores information indicating the calculated body motion score into the storage unit 140 as the body motion score information 143.

The agitation determination unit 153 makes the agitation determination based on the body motion score included in the body motion score information 143. The result of the agitation determination made by the agitation determination unit 153 is used when the labeling unit 154 labels the vital data.

For example, the agitation determination unit 153 includes a first threshold value, and a second threshold value having a value smaller than the first threshold value. Then, the agitation determination unit 153 makes the determination based on the body motion score, the first threshold value, and the second threshold value. For example, the agitation determination unit 153 determines that the target is agitated with respect to a period during which the body motion score is equal to or higher than the first threshold value. Further, the agitation determination unit 153 determines that the target is not agitated with respect to a period during which the body motion score is equal to or lower than the second threshold value. In this manner, the agitation determination unit 153 determines that the target is agitated if the body motion sore is equal to or higher than the first threshold value and also determines that the target is not agitated if the body motion score is equal to or lower than the second threshold value.

For example, FIG. 6 illustrates an example of the processing by the agitation determination unit 153 when the first threshold value and the second threshold value are 0.7 and 0.3, respectively. As illustrated in FIG. 6, the agitation determination unit 153 determines that the target is agitated with respect to a period during which the body motion score is equal to or higher than 0.7, which is the first threshold value. Further, the agitation determination unit 153 determines that the target is not agitated with respect to a period during which the body motion score is equal to or lower than 0.3, which is the second threshold value. Note that the agitation determination unit 153 does not determine whether the target is agitated with respect to a period during which the body motion score is higher than 0.3, which is the second threshold value, and lower than 0.7, which is the first threshold value. In other words, the agitation determination unit 153 determines that the body motion score does not satisfy the criterion for determining that the patient is agitated or not agitated.

The labeling unit 154 generates the supervisory data for the machine learning by labeling the vital data included in the sensing data 142 based on the result of the agitation determination made by the agitation determination unit 153. In the case where the sensing data 142 includes a plurality of types of vital data, the labeling unit 154 may label each piece of vital data.

For example, the labeling unit 154 assigns a label indicating that the agitation is present with respect to the period in the time-series vital data during which the agitation determination unit 153 determines that the target is agitated. Further, the labeling unit 154 assigns a label indicating that the agitation is absent with respect to the period in the time-series vital data during which the agitation determination unit 153 determines that the target is not agitated. On the other hand, the labeling unit 154 assigns no label with respect to the period in the time-series vital data during which the agitation determination unit 153 does not make the agitation determination. The labeling unit 154 generates the supervisory data by assigning the label with respect to partial periods in the time-series vital data during which the body motion score at the corresponding time satisfies the predetermined criterion in this manner by way of example.

The vital model training unit 155 generates the vital model by carrying out the machine learning using the supervisory data generated as a result of the labeling by the labeling unit 154. For example, the vital model training unit 155 carries out the machine learning using a support vector machine (SVM), a neural network, or the like. Then, the vital model training unit 155 stores the generated vital model into the storage unit 140 as the vital model 144.

As described above, the data in the time-series vital data with respect to the periods during which the body motion score satisfies the predetermined criterion such as the period during which the body motion score is equal to or higher than the first threshold value and the period during which the body motion score is equal to or lower than the second threshold value (labeled with the presence of the agitation and labeled with the absence of the agitation) is employed as the supervisory data. Therefore, the vital model training unit 155 can also be stated to carry out the training using a part of the vital data. Now, the period during which the body motion score is equal to or higher than the first threshold value is a period during which it is clear that the target is agitated, and the period during which the body motion score is equal to or lower than the second threshold value is a period during which it is clear that the target is not agitated. The accuracy of the determination using the generated vital model can be increased by carrying out the training using only the period during which it is clear that the agitation occurs and the period during which it is clear that the agitation does not occur as the supervisory data in this manner.

Note that, in the case where there is a plurality of pieces of vital data, this means that there are types of supervisory data (labels) according to the types of vital data. The vital model training unit 155 can generate one vital model to which the plurality of types of data is input according to the plurality of types of supervisory data. The vital model training unit 155 may carry out the machine learning for each type to generate a vital model according to each type.

Further, the vital model 144 generated by the vital model training unit 155 receives the information according to the vital data as an input, and outputs the vital score. Now, the vital score is an index for determining whether the patient is agitated and can be used to determine the predictive sign of agitation as described above. The vital score is, for example, a value from 0 to 1, inclusive. The vital score indicates that the patient exhibits the predictive sign of agitation as the value thereof is closer to 1, and indicates that the patient does not exhibit the predictive sign of agitation as the value thereof is closer to 0. The vital score may be an index expressed using two values that are 1 indicating that the patient exhibits the predictive sign of agitation and 0 indicating that the patient does not exhibit the predictive sign of agitation. The vital score may be an index indicating the intensity of the agitation, such as an index set to 2 for strong agitation and 1 for slight agitation.

Further, the vital model training unit 155 may carry out the training using the time-series data itself of the vital data such as the heart rate, or may carry out the training using various kinds of feature amounts calculated by subjecting the time-series data to processing for converting the time-series data into the feature amount such as averaging processing or differential processing.

The output unit 156 can output the vital model 144 generated by the vital model training unit 155 and the like to an external apparatus or the like.

This is an example of the configuration of the model training apparatus 100. Subsequently, examples of operations of the model training apparatus 100 will be described with reference to FIGS. 7 and 8.

FIG. 7 illustrates examples of operations of the model training apparatus 100. Referring to FIG. 7, the sensing data acquisition unit 151 acquires the body motion data and the vital data from the external apparatus such as the sensor apparatus via the communication I/F unit 130 (step S101). For example, the sensing data acquisition unit 151 acquires the body motion data and the vital data corresponding to the same time from the sensor apparatus that acquires both the body motion data and the vital data.

The body motion score calculation unit 152 calculates the body motion score using the body motion model 141 (step S102). For example, the body motion score calculation unit 152 calculates the time-series body motion score by inputting the time-series body motion data to the body motion model 141.

The agitation determination unit 153 makes the agitation determination based on the body motion score included in the body motion score information 143 (step S103). The details of the agitation determination processing will be described below.

The labeling unit 154 generates the supervisory data for the machine learning by labeling the vital data included in the sensing data 142 based on the result of the agitation determination made by the agitation determination unit 153 (step S104). For example, the labeling unit 154 assigns the label indicating that the agitation is present with respect to the period in the time-series vital data during which the agitation determination unit 153 determines that the target is agitated. Further, the labeling unit 154 assigns the label indicating that the agitation is absent with respect to the period in the time-series vital data during which the agitation determination unit 153 determines that the target is not agitated. On the other hand, the labeling unit 154 assigns no label with respect to the period in the time-series vital data during which the agitation determination unit 153 does not make the agitation determination.

The vital model training unit 155 generates the vital model by carrying out the machine learning using the supervisory data generated as a result of the labeling by the labeling unit 154 (step S105). For example, the vital model training unit 155 carries out the machine learning using a support vector machine (SVM), a neural network, or the like.

This is an example of the configuration of the model training apparatus 100. Note that the model training apparatus 100 can output the generated vital model 144 to an external apparatus or the like.

Subsequently, the details of the processing in step S103 illustrated in FIG. 7 will be described with reference to FIG. 8.

FIG. 8 illustrates further detailed one example of the processing by the agitation determination unit 153. Referring to FIG. 8, if the body motion score is equal to or higher than the first threshold value (YES in step S201), the agitation determination unit 153 determines that the target is agitated (step S202).

On the other hand, if the body motion score is lower than the first threshold value (NO in step S202), the agitation determination unit 153 determines whether the body motion score is equal to or lower than the second threshold value (step S203). If the body motion score is equal to or lower than the second threshold value (YES in step S203), the agitation determination unit 153 determines that the target is not agitated (step S204). On the other hand, if the body motion score is higher than the second threshold value (NO in step S203), the agitation determination unit 153 determines that the body motion score does not satisfy the criterion for determining that the patient is agitated or not agitated.

This is an example of the processing in step S103. Note that the processing in step S103 may be performed in an order different from the above-described example.

In this manner, the model training apparatus 100 includes the body motion score calculation unit 152, the agitation determination unit 153, the labeling unit 154, and the vital model training unit 155. Due to such a configuration, the agitation determination unit 153 can make the agitation determination based on the body motion score calculated by the body motion score calculation unit 152. Further, the labeling unit 154 can generate the supervisory data for the machine learning by labeling the vital data based on the result of the agitation determination made by the agitation determination unit 153. Accordingly, the efficient generation of the supervisory data can be achieved. Further, the vital model training unit 155 can carry out the machine learning using the supervisory data generated by the labeling unit 154. Accordingly, the supervisory data can be generated and the vital model 144 can be generated without performing processing that takes time and effort, such as manual labeling. In other words, according to the above-described configuration, the vital model 144 adapted to a new environment can be generated without taking time and effort. As a result, this configuration makes it possible to achieve a system that determines the agitation without taking time and effort.

Further, the model training apparatus 100 carries out the training while employing only the data in the time-series vital data with respect to the period during which the body motion score satisfies the predetermined criterion as the supervisory data. The accuracy of the determination using the generated vital model can be increased by carrying out the training using only a part of the vital data that satisfies the predetermined criterion in this manner. Note that the predetermined criterion is, for example, a period during which the body motion score satisfies the predetermined criterion, such as the period during which the body motion score is equal to or higher than the first threshold value or the period during which the body motion score is equal to or lower than the second threshold value, as described above. Note that the predetermined criterion may be a criterion different from the example indicated in the present exemplary embodiment, such as a period during which an absolute value of the body motion data such as the acceleration is equal to or greater than a predetermined value.

Note that the supervisory data generated by the labeling unit 154 of the model training apparatus 100 may be utilized when the already generated vital model 144 is retrained. In other words, the model training apparatus 100 may be configured to retrain the vital model 144 using the supervisory data generated by the labeling unit 154.

Now, the model training apparatus 100 can carry out the retraining using both previously acquired supervisory data and the supervisory data generated by the labeling unit 154. In other words, the model training apparatus 100 can carry out the retraining using supervisory data or the like used when the vital model 144 has been generated previously and the supervisory data generated by the labeling unit 154. Alternatively, the model training apparatus 100 may carry out the retraining using only the supervisory data generated by the labeling unit 154 without using previously acquired supervisory data. Only the supervisory data generated by the labeling unit 154 may be used or data different from the supervisory data generated by the labeling unit 154 may also be used in the retraining by the model training apparatus 100 in this manner by way of example. Note that which method to use from the above-described exemplary methods can be determined based on, for example, the number of pieces of supervisory data generated by the labeling unit 154 or the accuracy of the vital model 144. For example, the model training apparatus 100 can be configured to carry out the retraining also using previously acquired supervisory data if the number of pieces of supervisory data generated by the labeling unit 154 is equal to or smaller than a predetermined number. On the other hand, for example, the model training apparatus 100 can be configured to carry out the retraining using only the supervisory data generated by the labeling unit 154 if the number of pieces of supervisory data generated by the labeling unit 154 is greater than the predetermined number. Note that the retraining using only the supervisory data generated by the labeling unit 154 may be carried out if, for example, the number of pieces of supervisory data generated by the labeling unit 154 is greater than the predetermined number and the accuracy of the vital model 144 is deteriorated to fall below a setting value. The model training apparatus 100 may be configured to, for example, perform each of the above-described plurality of retraining methods and employ the vital model 144 achieving excellent accuracy.

Further, the agitation determination unit 153 may make the agitation determination using only one type of threshold value. In other words, the model training apparatus 100 may be configured to generate the supervisory data without making the selection from the time-series vital data based on the predetermined criteria and carry out the retraining based on the generated supervisory data.

Further, the configuration of the model training apparatus 100 is not limited to the example described with reference to FIG. 1. For example, FIG. 9 illustrates another example of the configuration of the model training apparatus 100. Referring to FIG. 9, the arithmetic processing unit 150 of the model training apparatus 100 can include, for example, attribute information acquisition unit 157.

The attribute information acquisition unit 157 acquires attribute information of the target. For example, the attribute information acquisition unit 157 acquires medical record information of the target from an external apparatus or the like, and acquires attribute information such as an age, a gender, and a paralysis state.

The attribute information acquired by the attribute information acquisition unit 157 can be utilized, for example, when the model training apparatus 100 adjusts the first threshold value and/or the second threshold value and when the model training apparatus 100 determines whether to carry out the training. For example, the agitation determination unit 153 can adjust the first threshold value and/or the second threshold value based on the attribute information. In other words, the period used for the training can be changed based on the attribute information. Further, whether to use the vital data for the training may be determined based on the attribute information. For example, when the patient is determined to clearly have an unusual symptom based on the attribute information, such as being in a state requiring an absolute bed rest or being in a state that both the hands are paralyzed and cannot be moved, the vital model training unit 155 or the like can determine not to carry out the training using the vital data resulting from the measurement of the target having the unusual symptom. The attribute information acquired by the attribute information acquisition unit 157 may be utilized in processing different from the above-described examples, such as being used when the body motion score calculation unit 152 determines whether to calculate the body motion score.

Note that FIGS. 1 and 9 illustrate the example in which the functions as the model training apparatus 100 are realized by one information processing apparatus. However, the functions as the model training apparatus 100 may be realized by, for example, a plurality of information processing apparatuses connected via a network. For example, the model training apparatus 100 may be configured of a supervisory data generation apparatus including the sensing data acquisition unit 151, the body motion score calculation unit 152, the labeling-purpose agitation determination unit 153, and the labeling unit 154, and a training apparatus that carries out the training using the supervisory data generated by the supervisory data generation apparatus.

### Second Exemplary Embodiment

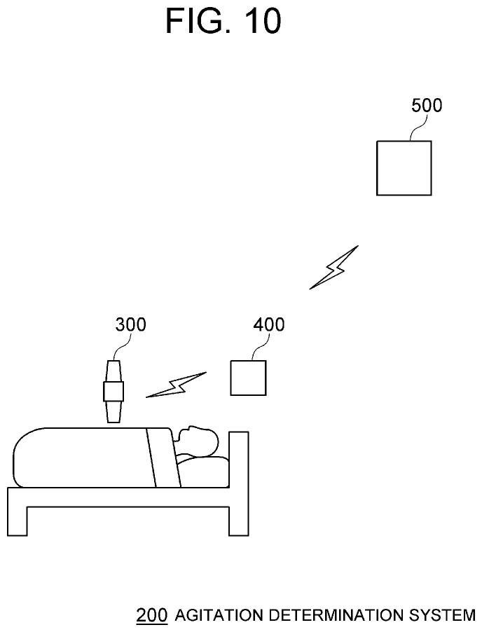

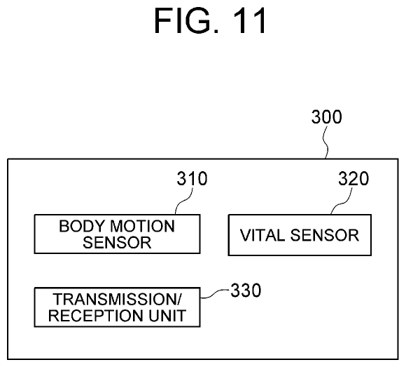

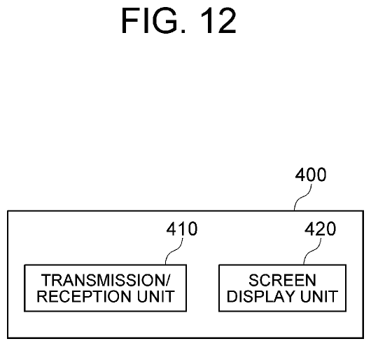

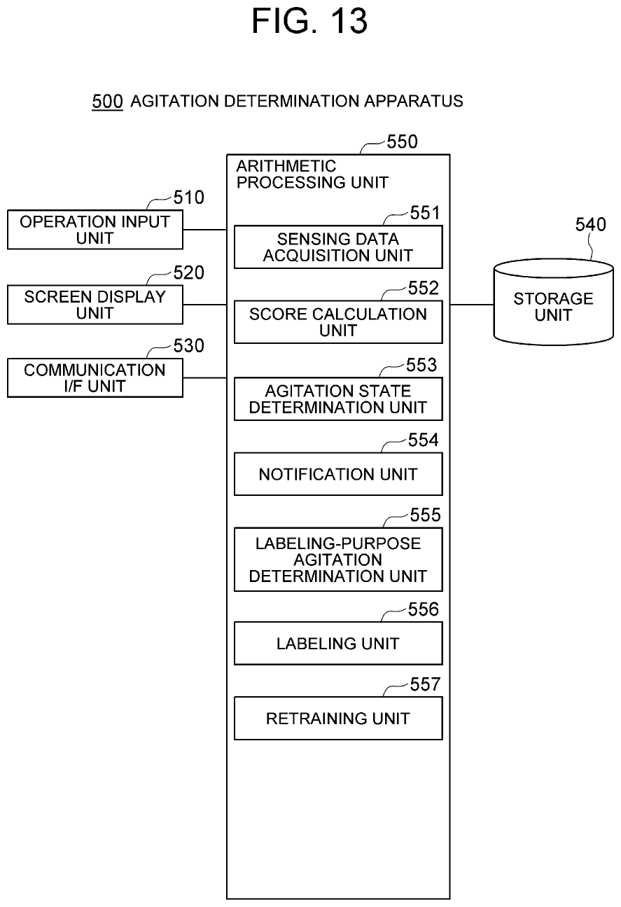

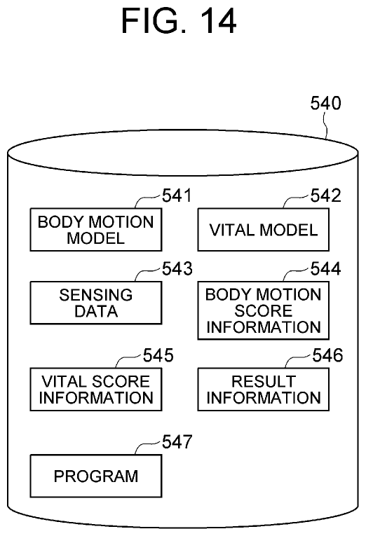

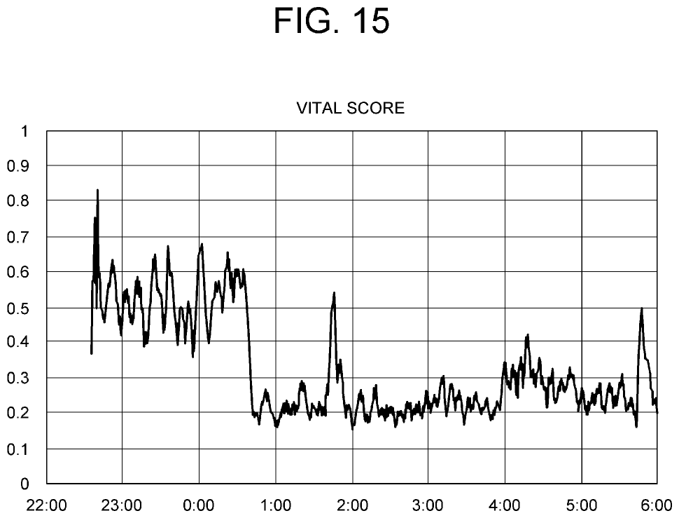

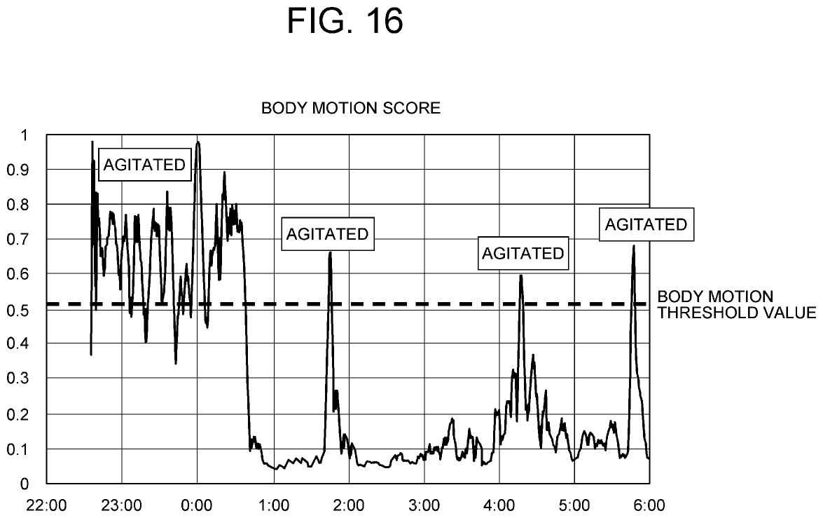

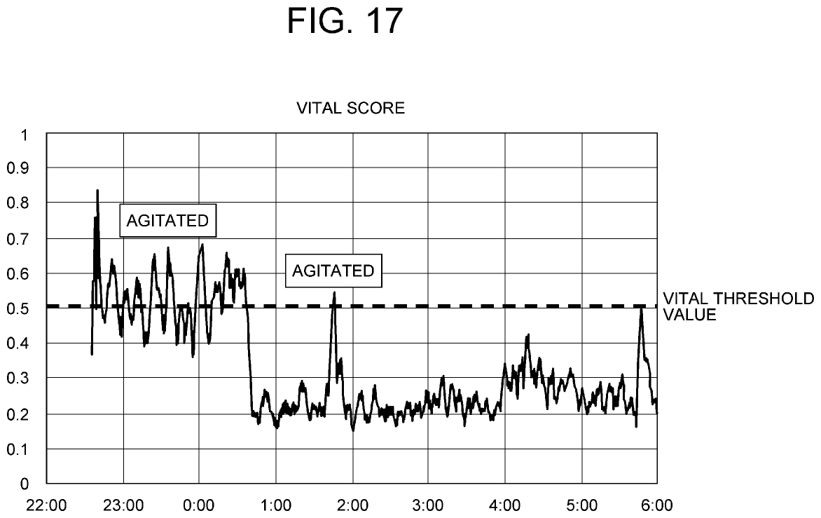

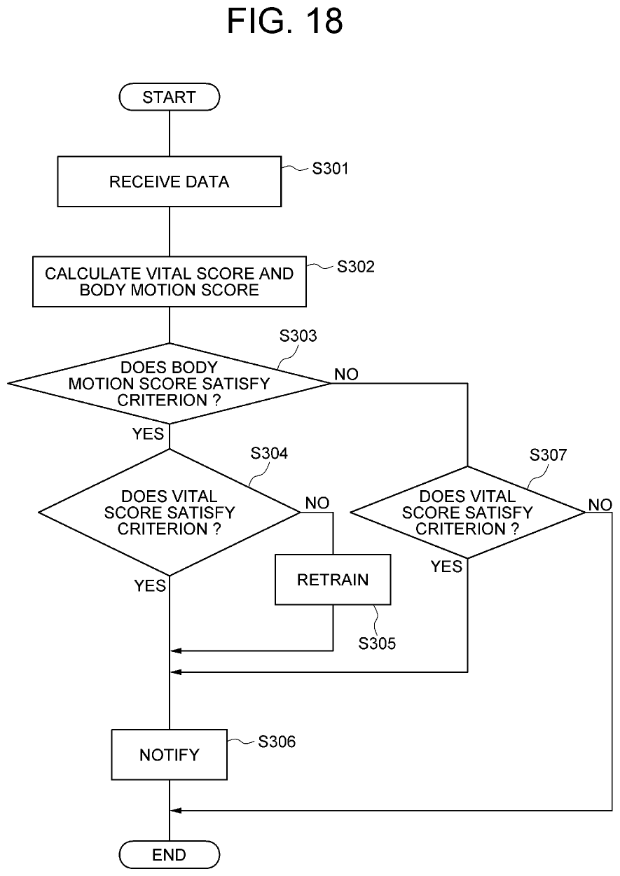

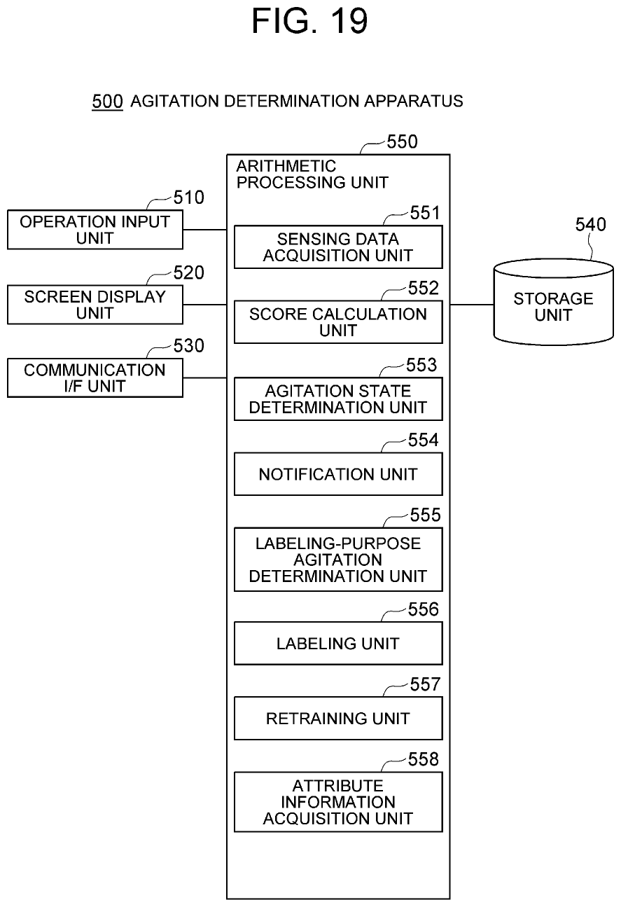

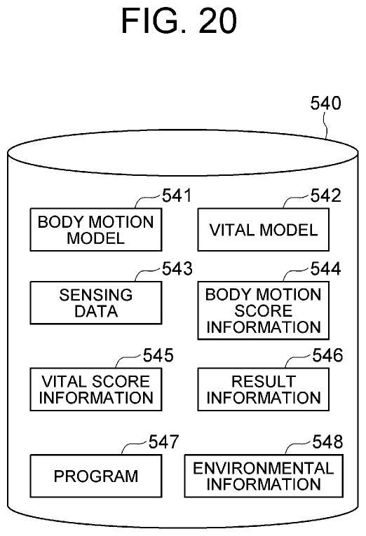

Subsequently, a second exemplary embodiment of the present disclosure will be described with reference to FIGS. 10 to 20. FIG. 10 is a diagram illustrating an example of the configuration of an agitation determination system 200. FIG. 11 is a block diagram illustrating an example of the configuration of a sensor apparatus 300. FIG. 12 is a block diagram illustrating an example of the configuration of a bed terminal 400. FIG. 13 is a block diagram illustrating an example of the configuration of an agitation determination apparatus 500. FIG. 14 illustrates one example of information stored in a storage unit 540. FIG. 15 is a diagram illustrating one example of a vital score included in vital score information 545. FIG. 16 is a diagram for illustrating an example of processing by an agitation state determination unit 553. FIG. 17 is a diagram for illustrating an example of processing by the agitation state determination unit. FIG. 18 is a flowchart illustrating examples of operations of the agitation determination apparatus 500. FIG. 19 is a block diagram illustrating another example of the configuration of the agitation determination apparatus 500. FIG. 20 illustrates one example of other information stored in the storage unit 540.

The second exemplary embodiment of the present disclosure will be described regarding the agitation determination system 200, which determines an agitation state of a patient set as a target wearing the sensor apparatus 300 based on data measured using the sensor apparatus 300. As will be described below, in the case of the present exemplary embodiment, the sensor apparatus 300 includes a body motion sensor 310, which measures body motion data such as an acceleration at a predetermined portion of the patient, and a vital sensor 320, which measures vital data that is information according to a state of the patient, such as a heart rate. Then, the agitation determination system 200 determines the agitation state of the patient based on a body motion score calculated based on the body motion data that the body motion sensor 310 acquires by measuring it, and a vital score calculated based on the vital data that the vital sensor 320 acquires by measuring it. By making the determination based on the body motion score and the vital score, the agitation determination system 200 can determine a predictive sign of agitation and also determine that the patient is in an agitated state without overlooking it even when the patient transitions to the agitated state without exhibiting the predictive sign of agitation. Further, the agitation determination system 200 can generate supervisory data for retraining a vital model 542 using the body motion score calculated based on the body motion data. Then, the agitation determination system 200 can retrain the vital model 542 using the generated supervisory data.

Note that the agitation determination system 200, which will be described in the present exemplary embodiment, can be utilized in various scenes, such as an acute hospital, a convalescent hospital, a care facility, and monitoring at home. In the following description, the present exemplary embodiment will be described citing an example when the agitation determination system 200 is utilized in a hospital such as an acute hospital or a convalescent hospital. Note that the agitation determination system 200 may be utilized under a condition requiring the agitation determination that is different from the above-described examples.

FIG. 10 illustrates an example of the configuration of the agitation determination system 200. Referring to FIG. 10, the agitation determination system 200 includes, for example, the sensor apparatus 300, the bed terminal 400, and the agitation determination apparatus 500. As illustrated in FIG. 10, the sensor apparatus 300 and the bed terminal 400 are mutually communicably connected using short-range wireless communication such as Bluetooth (registered trademark), wired communication, or the like. Further, the bed terminal 400 and the agitation determination apparatus 500 are mutually communicably connected using short-range wireless communication such as Wi-Fi (registered trademark), wired communication, or the like. The bed terminal 400 and the agitation determination apparatus 500 may be connected via a relay apparatus such as a wireless base station.

Note that the number of sensor apparatuses 300, the number of bed terminals 400, and the number of agitation determination apparatuses 500 included in the agitation determination system 200 are not limited to the numbers exemplarily illustrated in FIG. 10. For example, the agitation determination system 200 may include a plurality of sensor apparatuses 300, a plurality of bed terminals 400, and/or a plurality of agitation determination apparatuses 500.

The sensor apparatus 300 is an apparatus including a sensor worn on at least one portion of the patient set as the target. The sensor apparatus 300 measures the body motion data, which is a physical amount regarding the motion of the patient, and also measures the vital data, which is a physical amount regarding the vital of the patient. FIG. 11 illustrates an example of the configuration of the sensor apparatus 300. Referring to FIG. 11, the sensor apparatus 300 includes, for example, the body motion sensor 310, the vital sensor 320, and a transmission/reception unit 330. For example, the sensor apparatus 300 can realize each of the above-described processing units by hardware. The sensor apparatus 300 may realize each of the above-described processing units through execution of a program stored in a storage device by an arithmetic device such as a CPU.

The body motion sensor 310 acquires time-series body motion data by measuring the body motion data, which is the physical amount regarding the motion of the patient. For example, the body motion sensor 310 is at least one of an acceleration sensor, a gyro sensor, an angular sensor, a microphone, or the like, and measures the body motion data such as an acceleration, an angular velocity, and/or an angle at a predetermined portion such as the arm, body, or foot of the patient wearing the sensor apparatus 300, and/or a vocal volume.

In the case of the present exemplary embodiment, the body motion sensor 310 includes the acceleration sensor, and measures the acceleration at the predetermined portion of the patient. The body motion sensor 310 may measure body motion data different from the above-described examples as described above.

The vital sensor 320 acquires time-series vital data by measuring the vital data, which is the physical amount regarding the vital of the patient. For example, the vital sensor 320 is at least one of a heart rate sensor, a blood pressure sensor, a respiratory rate sensor, a body temperature sensor, a blood oxygen saturation level sensor, or the like, and measures the vital data such as a heart rate, a blood pressure value, a respiratory rate, a body temperature, a skin temperature, a blood flow rate, and/or a blood oxygen saturation level of the patient wearing the sensor apparatus 300.

In the case of the present exemplary embodiment, the vital sensor 320 includes the heart rate sensor, and measures the heart rate of the patient. The vital sensor 320 may measure vital data different from the above-described examples as described above.

The transmission/reception unit 330 includes an antenna or the like, and transmits and receives data between the sensor apparatus 300 and the bed terminal 400. For example, the transmission/reception unit 330 transmits the body motion data acquired by the body motion sensor 310 and the vital data acquired by the vital sensor 320 to the bed terminal 400. Further, the transmission/reception unit 330 can transmit patient identification information for identifying the patient such as identification information provided to the sensor apparatus 300 in advance in association with the above-described body motion data and vital data.

This is an example of the configuration of the sensor apparatus 300. Note that the sensor apparatus 300 may be configured of one device or may be configured of a plurality of devices. For example, the sensor apparatus 300 can be configured of one device having functions as the body motion sensor 310 and the vital sensor 320. Alternatively, the sensor apparatus 300 may be configured of a plurality of devices such as a device having a function as the body motion sensor 310 and a device having a function as the vital sensor 320. The body motion sensor 310 may be configured of a plurality of devices, such as the acceleration sensor and the angular velocity sensor. Further, the vital sensor 320 may be configured of a plurality of devices, such as the heart rate sensor and the skin/body temperature sensor. Note that, in the case where the sensor apparatus 200 is configured of a plurality of devices, the plurality of devices may each include a transmission/reception unit that transmits and receives data.

The bed terminal 400 is an information processing apparatus set up in advance at, for example, a predetermined location such as around the bed on which the patient stays. The bed terminal 400 is, for example, a smartphone, and has a screen display function. The bed terminal 400 may be an apparatus different from the smartphone. Note that the bed terminal 300 is a terminal set up at a predetermined location where the patient should stay or a predetermined location based on which a range where the patient should stay is defined, and is not limited to the terminal set up around the bed.

FIG. 12 illustrates an example of the configuration of the bed terminal 400. Referring to FIG. 12, the bed terminal 400 includes, for example, a transmission/reception unit 410 and a screen display unit 420. For example, the bed terminal 400 can realize each of the above-described processing units by hardware. The bed terminal 400 may realize each of the above-described processing units through execution of a program stored in a storage device by an arithmetic device such as a CPU.

The transmission/reception unit 410 includes an antenna or the like, and transmits and receives data between the bed terminal 400, and the sensor apparatus 300 and the agitation determination apparatus 500. The transmission/reception unit 410 receives, for example, the body motion data, the vital data, and the patient identification information transmitted from the sensor apparatus 300. Then, the transmission/reception unit 410 transmits, for example, the body motion data, the vital data, and the patient identification information received from the sensor apparatus 300 to the agitation determination apparatus 500. Further, the transmission/reception unit 410 can receive information indicating a result of the determination about the agitation from the agitation determination apparatus 500.

The screen display unit 420 displays, for example, the body motion data, the vital data, the patient identification information, and the information indicating the result of the determination about the agitation received by the transmission/reception unit 410 on a screen. For example, the screen display unit 420 can display that the patient corresponding to the bed terminal 400 is in the agitated state or the like on the screen based on the received information indicating the result of the determination about the agitation or the like.

The agitation determination apparatus 500 is an information processing apparatus that makes the determination based on the body motion data and the vital data measured by the sensor apparatus 300. Further, the agitation determination apparatus 500 can retrain the vital model 542 using the supervisory data generated based on the vital data and the body motion data. For example, the agitation determination apparatus 500 is set up at a predetermined location such as a nurse station. The agitation determination apparatus 500 is, for example, an information processing apparatus such as a personal computer or a tablet used by medical staff such as a doctor or a nurse, a server set up in a hospital or the like, or a cloud server. The agitation determination apparatus 500 may be a combination of the information processing apparatus such as a personal computer or a tablet, and the server and/or the like.

FIG. 13 illustrates an example of the configuration of the agitation determination apparatus 500. Referring to FIG. 13, the agitation determination apparatus 500 includes, for example, an operation input unit 510, a screen display unit 520, a communication I/F unit 530, the storage unit 540, and an arithmetic processing unit 550 as main constituent elements thereof.

The operation input unit 510, the screen display unit 520, and the communication I/F unit 530 may be configured similarly to the operation input unit 110, the screen display unit 120, and the communication I/F unit 130 described in the first exemplary embodiment. Therefore, the descriptions thereof will be omitted here.

The storage unit 540 is a storage device such as a hard disk or a memory. FIG. 14 illustrates one example of information stored in the storage unit 540. As illustrated in FIG. 14, the storage unit 540 stores processing information required for various kinds of processing by the arithmetic processing unit 550, and a program 547 therein. The program 547 realizes the various kinds of processing units by being read in and executed by the arithmetic processing unit 550. The program 547 is read in from an external apparatus or a recording medium in advance via a data input/output function such as the communication I/F unit 530, and is stored in the storage unit 540. Examples of main information stored in the storage unit 540 include a body motion model 541, the vital model 542, sensing data 543, body motion score information 544, vital score information 545, and result information 546.

The body motion model 541 may be similar to the body motion model 141 described in the first exemplary embodiment. The body motion model 541 may include one type of model or may include a plurality of types of models according to the type of the body motion data.

The vital model 542 corresponds to, for example, the vital model 144 generated by the model training apparatus 100 described in the first exemplary embodiment. The vital model 542 may be generated by carrying out machine learning using data generated by labeling previously measured vital data with the presence or absence of the agitation, for example, manually as the supervisory data in an external apparatus different from the model training apparatus 100 described in the first exemplary embodiment. For example, the vital model 542 is acquired from the external apparatus such as the model training apparatus 100 via the communication I/F unit 530 or the like, and is stored in the storage unit 540. Note that the vital model 542 may include one type of model or may include a plurality of types of models according to the type of the vital data.

The sensing data 543 includes the data measured by the sensor apparatus 300. For example, the sensing data 543 includes the patient identification information, the body motion data, and the vital data in association with one another. In the case of the present exemplary embodiment, the body motion data includes the time-series data of the acceleration as described above. Further, the vital data includes the time-series data of the heart rate. The body motion data and the vital data included in the sensing data 543 may also be similar to the body motion data and the vital data described in the first exemplary embodiment.

The body motion score information 544 includes the body motion score, which is an index for determining whether the patient is agitated. For example, the body motion score information 544 includes the patient identification information and the body motion score in association with each other. The body motion score included in the body motion score information 544 may also be similar to the body motion score described in the first exemplary embodiment.

The vital score information 545 includes the vital score, which is an index for determining the predictive sign of agitation. For example, the vital score information 545 includes the patient identification information and the vital score in association with each other.

FIG. 15 illustrates one example of the vital score calculated by a score calculation unit 552 based on the vital data like the example illustrated in FIG. 4 in the first exemplary embodiment. In the case of FIG. 15, an x axis represents time and a y axis represents the vital score. As illustrated in FIG. 15, the vital score is expressed by, for example, a value from 0 to 1, inclusive. The vital score indicates that the patient exhibits the predictive sign of agitation as the value thereof is closer to 1, and indicates that the patient does not exhibit the predictive sign of agitation as the value thereof is closer to 0.

The result information 546 includes information indicating the result of the determination made by the agitation state determination unit 553 based on the body motion score information 544 and the vital score information 545. For example, the result information 546 includes the patient identification information and the information indicating the result of the determination.

The arithmetic processing unit 550 includes a microprocessor such as an MPU and a peripheral circuit thereof. The arithmetic processing unit 550 reads in the program 547 from the storage unit 540 and executes it, thereby causing the above-described hardware and the program 547 to cooperate with each other to realize various kinds of processing units. Examples of main processing units realized by the arithmetic processing unit 450 include a sensing data acquisition unit 551, the score calculation unit 552, the agitation state determination unit 553, a notification unit 554, a labeling-purpose agitation determination unit 555, a labeling unit 556, and a retraining unit 557. Note that the score calculation unit 552, the agitation state determination unit 553, and the notification unit 554 among the above-described processing units mainly perform processing for determining whether the patient is in the agitated state. Further, the labeling-purpose agitation determination unit 555, the labeling unit 556, and the retraining unit 557 among the above-described processing units mainly perform processing for retraining the vital model 542.

The sensing data acquisition unit 551 acquires the body motion data, the vital data, the patient identification information, and the like transmitted from the bed terminal 400 via the communication IF unit 530. Then, the sensing data acquisition unit 551 stores the acquired body motion data and vital data into the storage unit 540 in association with the patient identification information as the sensing data 543.

The score calculation unit 552 calculates the body motion score using the body motion model 541 and also calculates the vital score using the vital model 542.

For example, the score calculation unit 552 acquires the body motion data by referring to the sensing data 543. Further, the score calculation unit 552 inputs the acquired data to the body motion model 541 to calculate the body motion score at each time. After that, the score calculation unit 552 stores information indicating the calculated body motion score into the storage unit 540 as the body motion score information 544.

For example, the score calculation unit 552 acquires the vital data by referring to the sensing data 543. Further, the score calculation unit 552 inputs the acquired data to the vital model 542 to calculate the vital score at each time like the example illustrated in FIG. 15. After that, the score calculation unit 552 stores information indicating the calculated vital score into the storage unit 540 as the vital score information 545.

Note that the score calculation unit 552 may input the time-series data itself to the body motion model 541 and the vital model 542, or may input various kinds of feature amounts calculated by subjecting the time-series data to processing for converting the time-series data into the feature amount such as averaging processing or differential processing to the body motion model 541 and the vital model 542.

The agitation state determination unit 553 determines the agitation state of the patient based on the body motion score included in the body motion score information 544 and the vital score included in the vital score information 545. For example, the agitation state determination unit 553 determines whether the patient is agitated or whether the patient exhibits the predictive sign of agitation as the agitation state of the patient. Then, the agitation state determination unit 553 stores the result of the determination into the storage unit 540 as the result information 546. For example, the agitation state determination unit 553 stores the information indicating the result of the determination about the agitation state into the storage unit 540 as the result information 546. Further, the agitation state determination unit 553 can determine whether to retrain the vital model 542 according to the result of the determination about the agitation state based on the body motion score and the vital score.

For example, the agitation state determination unit 553 includes a body motion threshold value to be compared with the body motion score and a vital threshold value to be compared with the vital score in advance. Then, the agitation state determination unit 553 makes the determination based on the body motion score, the vital score, the body motion threshold value, and the vital threshold value. For example, the agitation state determination unit 553 determines that the patient is agitated if the body motion score is equal to or higher than the body motion threshold value. The agitation state determination unit 553 makes the determination based on the body motion score in this manner, thereby being able to determine that the patient is agitated when the patient is actually agitated. Further, for example, the agitation state determination unit 553 determines that the patient is agitated or the patient exhibits the predictive sign of agitation if the vital score is equal to or higher than the vital threshold value. Further, for example, the agitation state determination unit 553 determines that the patient exhibits the predictive sign of agitation if the vital score is equal to or higher than the vital threshold value even when the body motion score is lower than the body motion threshold value. The agitation state determination unit 553 may determine that the patient is in a normal state if the body motion score is equal to or lower than the body motion threshold value and the vital score is equal to or lower than the vital threshold value. In this manner, the agitation state determination unit 553 determines whether the patient is agitated or whether the patient exhibits the predictive sign of agitation based on the body motion score and the vital score.

FIGS. 16 and 17 illustrate an example of processing by the agitation state determination unit 553. For example, referring to FIG. 16, the body motion score becomes equal to or higher than the body motion threshold value during a period from 22:30 to slightly before 1:00, slightly before 2:00, around 4:30, and around 6:00. Accordingly, the agitation state determination unit 553 determines that the patient is agitated with respect to these periods. Further, referring to FIG. 17, the vital score becomes equal to or higher than the vital threshold value during the period from 22:30 to slightly before 1:00 and slightly before 2:00. Accordingly, the agitation state determination unit 553 determines that the patient exhibits the predictive sign of agitation with respect to these periods.

Note that the values of the body motion threshold value and the vital threshold value may be set in any manner. For example, in the case of FIGS. 16 and 17, the values of the body motion threshold value and the vital threshold value are set to the same value. However, the values of the body motion threshold value and the vital threshold value may be values different from each other. Further, the body motion threshold value and the vital threshold value may be determined as appropriate according to, for example, attribute information of the patient, which will be described below.

Further, the agitation state determination unit 553 may consistently determine that the patient is agitated if the body motion score is equal to or higher than the body motion threshold value or if the vital score is equal to or higher than the vital threshold value. According to such a configuration, the medical staff can be notified of an abnormality in the patient without missing any of it.

Further, if being unable to use any one of the body motion score and the vital score to determine the agitation state of the patient, the agitation state determination unit 553 may determine the agitation state of the patient using the other of the scores alone. Now, being unable to use the body motion score to determine the agitation state of the patient includes, for example, being unable to acquire the body motion data by the sensor apparatus 300, and being unable to acquire the body motion data of the patient by the sensing data acquisition unit 551 due to a communication trouble or the like. Further, being unable to use the vital score to determine the agitation state of the patient includes, for example, being unable to acquire the vital data by the sensor apparatus 300, and being unable to acquire the vital data of the patient by the sensing data acquisition unit 551 due to a communication trouble or the like.

Further, when the body motion score is equal to or higher than the body motion threshold value and the vital score is lower than the vital threshold value, it is considered that an oversight may occur in the agitation determination based on the vital score. Therefore, the agitation state determination unit 553 determines to retrain the vital model 542 if the body motion score is equal to or higher than the body motion threshold value and the vital score is lower than the vital threshold value. For example, the retraining processing by the labeling-purpose agitation determination unit 555, the labeling unit 556, and the retraining unit 557 is performed according to this determination.

The notification unit 554 outputs the result of the determination made by the agitation state determination unit 553, for example, when the agitation state determination unit 553 determines that the patient is agitated. For example, when the agitation state determination unit 553 determines that the patient is agitated or determines that the patient exhibits the predictive sign of agitation, the notification unit 554 can display the information indicating the result of the determination on the screen display unit 520 together with the patient identification information of the patient. Further or alternatively, the notification unit 554 can transmit the information indicating the result of the determination and the patient identification information of the patient to an external apparatus such as the bed terminal 400 relating to this patient or the mobile terminal carried by the nurse in charge of this patient. Note that the notification unit 554 may issue a notification in a manner different from the above-described example, such as lighting a lamp at an entrance of a room in which the patient is hospitalized.

Note that the notification unit 554 may change the display content and/or the notification content according to a difference in the determination, i.e., whether the patient is determined to be agitated or determined to exhibit the predictive sign of agitation. For example, the notification unit 554 may use a method such as lighting a lamp in a different color according to the determination result when lighting the lamp at the entrance of the room in which the patient is hospitalized.

The labeling-purpose agitation determination unit 555 makes an agitation determination for carrying out labeling based on the body motion score included in the body motion score information 544 according to the determination to carry out the retraining by the agitation state determination unit 553. The processing by the labeling-purpose agitation determination unit 555 may be similar to the processing performed by the agitation determination unit 153 described in the first exemplary embodiment.

The labeling unit 556 generates supervisory data for machine learning by labeling the vital data included in the sensing data 543 based on the result of the determination made by the labeling-purpose agitation determination unit 555. The processing by the labeling unit 556 may also be similar to the processing by the labeling unit 154 described in the first exemplary embodiment.

The retraining unit 557 generates the vital model 542 by carrying out the machine learning using the supervisory data generated as a result of the labeling by the labeling unit 556. For example, the retraining unit 557 carries out the machine learning using a support vector machine (SVM), a neural network, or the like, similarly to the vital model training unit 155 described in the first exemplary embodiment. Then, the retraining unit 557 stores the retrained vital model into the storage unit 540 as the vital model 542. Note that only the supervisory data generated by the labeling unit 556 may be used or data different from the supervisory data generated by the labeling unit 556 may also be used in the retraining by the retraining unit 557 similarly to the retraining exemplarily indicated in the first exemplary embodiment. The retraining unit 557 may be configured to perform processing similar to the processing exemplarily indicated in the first exemplary embodiment.

This is an example of the configuration of the agitation determination system 200. Subsequently, examples of operations of the agitation determination apparatus 500 will be described with reference to FIG. 18. Note that the order of the operations of the agitation determination apparatus 500 illustrated in FIG. 18 is one example, and the operations of the agitation determination apparatus 500 are not limited thereto.

The sensing data acquisition unit 551 acquires the body motion data, the vital data, the patient identification information, and the like transmitted from the bed terminal 400 via the communication IF unit 530 (step S301).

The score calculation unit 552 calculates the body motion score using the body motion model 541 and also calculates the vital score using the vital model 542 (step S302). For example, the score calculation unit 552 calculates the body motion score by inputting the body motion data to the body motion model 541. Further, the score calculation unit 552 calculates the vital score by inputting the vital data to the vital model 542.

The agitation state determination unit 553 determines whether the patient is in the agitated state based on the body motion score included in the body motion score information 544 and the vital score included in the vital score information 545. For example, if the body motion score is equal to or higher than the body motion threshold value (YES in step S303), the agitation state determination unit 553 determines that the patient is agitated. Further, if the body motion score is equal to or higher than the body motion threshold value and the vital score is lower than the vital threshold value (NO in step S304), the agitation state determination unit 553 determines to carry out the retraining. Accordingly, the vital model 542 is retrained (step S305). Note that the retraining processing can be performed by a method similar to the processing for generating the vital model described in the first exemplary embodiment.

If the patient is determined to be agitated, the notification unit 554 issues the notification (step S306). For example, the notification unit 554 can display that the patient is agitated on the screen display unit 520, and/or transmit the determination that the patient is agitated to the external apparatus such as the bed terminal 400 or the mobile terminal carried by the nurse in charge of the patient.

On the other hand, even if the body motion score is lower than the body motion threshold value (NO in step S303), if the vital score is equal to or higher than the vital threshold value (YES in step S307), the agitation state determination unit 553 determines that the patient exhibits the predictive sign of agitation. Then, the notification unit 554 issues the notification (step S306). On the other hand, if the body motion score is lower than the body motion threshold value (NO in step S303) and if the vital score is also lower than the vital threshold value (NO in step S307), the agitation state determination unit 553 determines that the patient is not agitated. In this case, the notification unit 554 does not issue the notification.

These are examples of the operations of the agitation determination apparatus 500.

In this manner, the agitation determination apparatus 500 includes the score calculation unit 552, which calculates the body motion score and the vital score, and the agitation state determination unit 553. Due to such a configuration, the agitation state determination unit 553 can make the determination based on the body motion score and also make the determination based on the vital score. As a result, when the patient is actually agitated, the agitation determination apparatus 500 can determine that the patient is agitated without missing even the agitation accompanied by a low vital score. Accordingly, the agitation determination apparatus 500 can make the determination without overlooking the agitation.

Further, the agitation determination apparatus 500 is configured to retrain the vital model 542 if the body motion score is equal to or higher than the body motion threshold value and the vital score is lower than the vital threshold value. When the body motion score is equal to or higher than the body motion threshold value and the vital score is lower than the vital threshold value, it is considered that an oversight may occur in the agitation determination based on the vital score. Therefore, the agitation determination apparatus 500 allows the accuracy of the vital model 542 to be increased as necessary by carrying out the retraining when the above-described criterion is satisfied.

Note that the present exemplary embodiment has been described assuming that the agitation determination apparatus 500 includes the agitation state determination unit 553 and the labeling-purpose agitation determination unit 555. However, the labeling unit 556 of the agitation determination apparatus 500 may carry out the labeling using the result of the determination made by the agitation state determination unit 553. In this case, the agitation determination apparatus 500 does not have to include the labeling-purpose agitation determination unit 555.

Further, in the case where the body motion model 441 includes a plurality of models, a plurality of body motion scores may be calculated. In the case where a plurality of body motion scores is calculated in this manner, the agitation state determination unit 453 can determine that the patient is in the agitated state, for example, if even any one of the plurality of body motion scores becomes equal to or higher than the body motion threshold value. The agitation state determination unit 453 may make the determination by a method different from the above-described example, such as determining the agitation if more than half of the plurality of body motion scores become equal to or higher than the body motion threshold value.

Further, in the case where the vital model 442 includes a plurality of models, a plurality of vital scores may be calculated. In the case where a plurality of vital scores is calculated in this manner, the agitation state determination unit 453 can determine that the patient exhibits the predictive sign of agitation, for example, if even any one of the plurality of types of vital scores becomes equal to or higher than the vital threshold value. The agitation state determination unit 453 may determine that the patient exhibits the predictive sign of agitation if the plurality of vital scores satisfies a predetermined criterion, such as more than half of them being equal to or higher than the vital threshold value, all of them being equal to or higher than the vital threshold value, or an average value thereof being equal to or higher than the vital threshold value.

Further, the configuration of the agitation determination apparatus 500 is not limited to the example described with reference to FIG. 19. For example, FIG. 19 illustrates another example of the configuration of the agitation determination apparatus 500. Referring to FIG. 19, the arithmetic processing unit 550 of the agitation determination apparatus 500 can include, for example, attribute information acquisition unit 558.

The attribute information acquisition unit 558 acquires the attribute information of the patient. For example, the attribute information acquisition unit 558 acquires medical record information of the patient from an external apparatus or the like, and acquires attribute information such as an age, a gender, and a paralysis state.

The attribute information acquired by the attribute information acquisition unit 558 can be utilized, for example, when a priority of the model or the score is determined. More specifically, the attribute information can be used to determine a priority serving as an index for determining which model or which score should be prioritized to make the determination, for example, in the case where the body motion model 441 or the vital model 442 includes a plurality of models or in the case where the plurality of body motion scores or vital scores is calculated. Note that the attribute information may be utilized at the time of the training processing (the retraining processing) similarly to the first exemplary embodiment.

For example, suppose that the body motion model 541 includes the acceleration model and the vocal volume model, and the vital model 542 includes the heart rate model. Further, suppose that the patient is determined to have hemiplegia at his/her hand and/or foot based on the attribute information acquired by the attribute information acquisition unit 558. In this case, the patient is expected to less move. In light thereof, the agitation state determination unit 553 can lower the priority of the acceleration model and raise the priorities of the vocal volume model and the heart rate model. On the other hand, if the patient is determined to be a patient capable of moving by him/herself to some degree based on the attribute information acquired by the attribute information acquisition unit 558, the agitation state determination unit 553 can, for example, set the same priority to all of the acceleration model, the vocal volume model, and the heart rate model. Note that the agitation state determination unit 553 may adjust the priority based on other attribute information.

Note that the above-described priority is an index indicating which model or which score should be prioritized to make the determination, for example, in the case where the body motion model 541 or the vital model 542 includes a plurality of models or in the case where a plurality of body motion scores or vital scores is calculated. The agitation state determination unit 553 can, for example, adjust the criterion when making the determination based on the priority. For example, if the patient is determined to have hemiplegia at his/her hand and/or foot based on the attribute information, the agitation state determination unit 553 can lower the priority of the acceleration model and raise the priories of the vocal volume model and the heart rate model, as described above. In this case, the agitation state determination unit 553 can make an adjustment of the criterion, such as making the determination using only the score based on the vocal volume model and the score based on the heart rate model, or adjusting the body motion threshold value to be compared with the score based on the acceleration model to a value different from the body motion threshold value to be compared with the score based on the vocal volume model. On the other hand, if the patient is determined to be a patient capable of moving by him/herself to some degree, the agitation state determination unit 553 can, for example, set the same priority to all of the acceleration model, the vocal volume model, and the heart rate model, as described above. In this case, the agitation state determination unit 553 can make an adjustment of the criterion, such as making the determination using all of the score based on the acceleration model, the score based on the vocal volume model, and the score based on the heart rate model, or setting the same value to all of the body motion threshold value and the vital threshold value. Note that the agitation state determination unit 553 may adjust the criterion in a manner different from the above-described examples.

Further, referring to FIG. 20, environmental information 548 can be stored in the storage unit 540 of the agitation determination apparatus 500.

The environmental information 548 indicates information such as a season and/or a temperature. The environmental information 548 is, for example, acquired from an external apparatus via the communication I/F unit 530 or input in advance using the operation input unit 510.

The information included in the environmental information 548 can also be utilized, for example, when the priority is determined similarly to the attribute information acquired by the attribute information acquisition unit 558. For example, suppose that the body motion model 541 includes the acceleration model and the vocal volume model, and the vital model 542 includes the heart rate model, similarly to the above-described example. In this case, if the season is determined to be winter (or the temperature is determined to be low) based on the environmental information 548, the agitation state determination unit 553 can, for example, raise the priories of the vocal volume model and the heart rate model and lower the priority of the acceleration model because, for example, the patient tends to be slow-moving or a heart disease (an abnormal heart rate) easily occurs. Accordingly, the agitation state determination unit 553 can, for example, make a correction to the body motion threshold value and/or the vital threshold value, such as adjusting the body motion threshold value to be compared with the score based on the acceleration model to a value different from the body motion threshold value to be compared with the score based on the vocal volume model.

The agitation determination apparatus 500 may include the environmental information 548 and/or the attribute information acquisition unit 558 in this manner by way of example.

Note that FIGS. 13 and 19 illustrate the example in which the functions as the agitation determination apparatus 500 are realized by one information processing apparatus. However, the functions as the agitation determination apparatus 500 may be realized by, for example, a plurality of information processing apparatuses connected via a network.

### Third Exemplary Embodiment

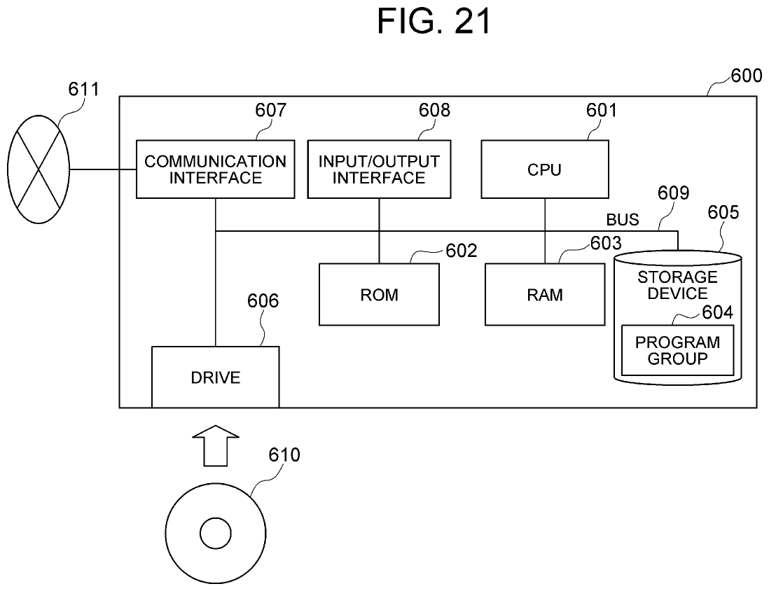

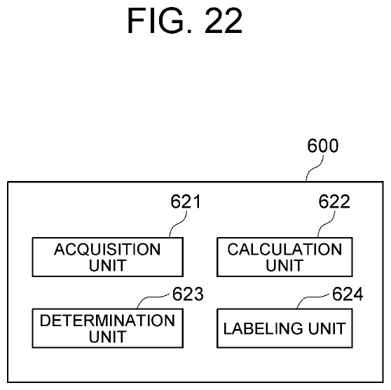

Next, a third exemplary embodiment of the present disclosure will be described with reference to FIGS. 21 and 22. The third exemplary embodiment will be described regarding an overview of the configuration of a supervisory data generation apparatus 600.

FIG. 21 illustrates an example of the hardware configuration of the supervisory data generation apparatus 600. Referring to FIG. 21, the supervisory data generation apparatus 600 has the following hardware configuration as one example.
Central Processing Unit (CPU) 601 (arithmetic unit)
Read Only Memory (ROM) 602 (storage unit)
Random Access Memory (RAM) 603 (storage unit)
Program group 604 to be loaded to the RAM 603
Storage device 605 storing therein the program group 604
Drive 606 that performs reading and writing on a recording medium 610 outside the information processing apparatus
Communication interface 607 connecting to a communication network 611 outside the information processing apparatus
Input/output interface 608 for performing input/output of data
Bus 609 connecting the constituent elements

Further, the supervisory data generation apparatus 600 can realize functions as an acquisition unit 621, a calculation unit 622, a determination unit 623, and a labeling unit 624 illustrated in FIG. 22 through acquisition of the program group 604 by the CPU 601 and execution thereof by this CPU 601. Note that the program group 604 is, for example, stored in the storage device 605 or the ROM 602 in advance, and loaded to the RAM 603 or the like and executed by the CPU 601 as needed. Alternatively, the program group 604 may be provided to the CPU 601 via the communication network 611, or may be stored in the recording medium 610 in advance and read out by the drive 606 and provided to the CPU 601.

Note that FIG. 21 illustrates an example of the hardware configuration of the supervisory data generation apparatus 600. The hardware configuration of the supervisory data generation apparatus 600 is not limited to the above-described example. For example, the supervisory data generation apparatus 600 may be configured of a part of the above-described configuration, such as not including the drive 606.

The acquisition unit 621 acquires body motion data according to a motion of a body of a target, and vital data according to a state of the body of the target.

The calculation unit 622 calculates a body motion score serving as an index for determining an agitation state based on the body motion data acquired by the acquisition unit 621.

The determination unit 623 determines whether the target is agitated based on the body motion score calculated by the calculation unit 622.

The labeling unit 624 labels the vital data based on a result of the determination made by the determination unit 623.

In this manner, the supervisory data generation apparatus 600 includes the acquisition unit 621, the calculation unit 622, the determination unit 623, and the labeling unit 624. Due to such a configuration, the labeling unit 624 can carry out labeling based on the result of the determination made by the determination unit 623 based on the body motion score calculated by the calculation unit 622. As a result, the supervisory data can be efficiently generated.

Note that the above-described supervisory data generation apparatus 600 can be realized by incorporating a predetermined program into a computer such as this supervisory data generation apparatus 600. More specifically, a program according to another exemplary embodiment of the present invention is a program for causing a computer to realize processing including acquiring body motion data according to a motion of a body of a target and vital data of the target, calculating a body motion score serving as an index for determining an agitation state based on the acquired body motion data, determining whether the target is agitated based on the body motion score, and labeling the vital data based on a result of the determining.

Further, a supervisory data generation method performed by the above-described training apparatus 600 is a method for causing a computer to acquire body motion data according to a motion of a body of a target and vital data of the target, calculate a body motion score serving as an index for determining an agitation state based on the acquired body motion data, determine whether the target is agitated based on the body motion score, and label the vital data based on a result of the determination.

Even the invention of the program (or a recording medium) or the supervisory data generation method configured in the above-described manner can also bring about advantageous effects equivalent to the above-described supervisory data generation apparatus 600, thereby achieving the above-described object of the present invention.

Further, examples of modifications of the supervisory data generation apparatus 600 include a training apparatus that trains a vital model. For example, the training apparatus includes an acquisition unit configured to acquire body motion data according to a motion of a body of a target and vital data of the target, a calculation unit configured to calculate a body motion score serving as an index for determining an agitation state based on the body motion data acquired by the acquisition unit, a determination unit configured to determine whether the target is agitated based on the body motion score, a labeling unit configured to label the vital data based on a result of the determination made by the determination unit, and a training unit configured to generate a vital model configured to output a vital score serving as an index for determining the agitation state by carrying out training using the labeled vital data as supervisory data.

Further, similarly, the examples of the modifications include an agitation determination apparatus. For example, the agitation determination apparatus includes an acquisition unit configured to acquire body motion data according to a motion of a body of a target and vital data of the target, a calculation unit configured to calculate a body motion score serving as an index for determining an agitation state based on the body motion data acquired by the acquisition unit and also calculate a vital score serving as an index for determining the agitation state based on the vital data acquired by the acquisition unit, a determination unit configured to determine to retrain a vital model used when the vital score is calculated based on the body motion score and the vital score calculated by the calculation unit, and a retraining unit configured to retrain the vital model according to a result of the determination unit.

### SUPPLEMENTARY NOTES

The whole or part of the exemplary embodiments disclosed above can be described as, but not limited to, the following supplementary notes. Hereinafter, outlines of the training apparatus and the like according to the present invention will be described. However, the present invention is not limited to the configurations described below.

### Supplementary Note 1

A supervisory data generation apparatus comprising:
an acquisition unit configured to acquire body motion data according to a motion of a body of a target and vital data of the target;
a calculation unit configured to calculate a body motion score serving as an index for determining an agitation state based on the body motion data acquired by the acquisition unit;
a determination unit configured to determine whether the target is agitated using the body motion score; and
a labeling unit configured to label the vital data based on a result of the determination made by the determination unit.

### Supplementary Note 2

The supervisory data generation apparatus according to supplementary note 1, wherein
the labeling unit carries out the labeling with respect to a partial period in the vital data during which the corresponding body motion score satisfies a predetermined criterion.

### Supplementary Note 3

The supervisory data generation apparatus according to supplementary note 1 or 2, wherein
the determination unit determines that the target is agitated with respect to a period during which the body motion score is equal to or higher than a first threshold value, and
the labeling unit carries out the labeling with respect to the period in the vital data during which the determination unit determines that the target is agitated.

### Supplementary Note 4

The supervisory data generation apparatus according to any one of supplementary notes 1 to 3, wherein
the determination unit determines that the target is not agitated with respect to a period during which the body motion score is equal to or lower than a second threshold value, and
the labeling unit carries out the labeling with respect to the period in the vital data during which the determination unit determines that the target is not agitated.

### Supplementary Note 5

The supervisory data generation apparatus according to any one of supplementary notes 1 to 4, further comprising:
an attribute information acquisition unit configured to acquire attribute information indicating an attribute of the target, wherein
the determination unit determines whether the target is agitated based on the attribute information acquired by the attribute information.

### Supplementary Note 6

The supervisory data generation apparatus according to any one of supplementary notes 1 to 5, further comprising:
an attribute information acquisition unit configured to acquire attribute information indicating an attribute of the target, wherein
the training unit determines whether to use the state information for training based on the attribute information acquired by the attribute information acquisition unit.

### Supplementary Note 7

A supervisory data generation method for causing a computer to:
acquire body motion data according to a motion of a body of a target and vital data of the target;
calculate a body motion score serving as an index for determining an agitation state based on the acquired body motion data;
determine whether the target is agitated based on the body motion score; and
label the vital data based on a result of the determination.

### Supplementary Note 8

A recording medium recording a program for causing a computer to realize processing including:
acquiring body motion data according to a motion of a body of a target and vital data of the target;
calculating a body motion score serving as an index for determining an agitation state based on the acquired body motion data;
determining whether the target is agitated based on the body motion score; and labeling the vital data based on a result of the determining by the determination unit.

### Supplementary Note 9

A training apparatus comprising:
an acquisition unit configured to acquire body motion data according to a motion of a body of a target and vital data of the target;
a calculation unit configured to calculate a body motion score serving as an index for determining an agitation state based on the body motion data acquired by the acquisition unit;
a determination unit configured to determine whether the target is agitated based on the body motion score;
a labeling unit configured to label the vital data based on a result of the determination made by the determination unit; and
a training unit configured to generate a vital model configured to output a vital score serving as an index for determining the agitation state by carrying out training using the labeled vital data as supervisory data.

### Supplementary Note 10

A training method for causing a computer to:
acquire body motion data according to a motion of a body of a target and vital data of the target;
calculate a body motion score serving as an index for determining an agitation state based on the acquired body motion data;
determine whether the target is agitated based on the body motion score;
label the vital data based on a result of the determination; and
generate a vital model configured to output a vital score serving as an index for determining the agitation state by carrying out training using the labeled vital data as supervisory data.

### Supplementary Note 11

A recording medium recording a program for causing a computer to realize processing including:
acquiring body motion data according to a motion of a body of a target and vital data of the target;
calculating a body motion score serving as an index for determining an agitation state based on the acquired body motion data;
determining whether the target is agitated based on the body motion score;
labeling the vital data based on a result of the determining; and
generating a vital model configured to output a vital score serving as an index for determining the agitation state by carrying out training using the labeled vital data as supervisory data.

### Supplementary Note 12

An agitation determination apparatus comprising:
an acquisition unit configured to acquire body motion data according to a motion of a body of a target and vital data according to a state of the body of the target;
a calculation unit configured to calculate a body motion score serving as an index for determining an agitation state based on the body motion data acquired by the acquisition unit and also calculate a vital score serving as an index for determining the agitation state based on the vital data acquired by the acquisition unit;
a determination unit configured to determine to retrain a vital model used when the vital score is calculated based on the body motion score and the vital score calculated by the calculation unit; and
a retraining unit configured to retrain the vital model according to a result of the determination unit.

### Supplementary Note 13

The agitation determination apparatus according to supplementary note 12, wherein
the determination unit determines to carry out the retraining in a case where the body motion score satisfies a predetermined criterion and the vital score does not satisfy a predetermined criterion.

### Supplementary Note 14

The agitation determination apparatus according to supplementary note 12 or 13, further comprising:
a labeling unit configured to generate supervisory data by labeling the vital data based on the result of the determination made by the determination unit, wherein
the retraining unit retrains the vital model by carrying out retraining using the supervisory data generated by the labeling unit.

### Supplementary Note 15

The agitation determination apparatus according to supplementary note 14, wherein
the labeling unit generates the supervisory data with respect to a part of the vital data.

### Supplementary Note 16

The agitation determination apparatus according to supplementary note 14 or 15, wherein
the labeling unit generates the supervisory data with respect to a partial period in the vital data during which the corresponding body motion score satisfies a predetermined criterion.

### Supplementary Note 17

A retraining method for causing a computer to:
acquire body motion data according to a motion of a body of a target and vital data according to a state of the body of the target;
calculate a body motion score serving as an index for determining an agitation state based on the acquired body motion data and also calculate a vital score serving as an index for determining the agitation state based on the acquired vital data;
determine to retrain a vital model used when the vital score is calculated based on the calculated body motion score and vital score; and
retrain the vital model according to a result of the determination.

### Supplementary Note 18

A recording medium recording a program for causing a computer to realize processing including:
acquiring body motion data according to a motion of a body of a target and vital data according to a state of the body of the target;
calculating a body motion score serving as an index for determining an agitation state based on the acquired body motion data and also calculating a vital score serving as an index for determining the agitation state based on the acquired vital data;
determining to retrain a vital model used when the vital score is calculated based on the calculated body motion score and vital score; and
retraining the vital model according to a result of the determining.

Note that the program disclosed in each of the above-described exemplary embodiments and the supplementary notes is, for example, stored in a storage device or recorded in a computer-readable recording medium. For example, the recording medium is a portable medium such as a flexible disk, an optical disk, a magneto-optical disk, or a semiconductor memory.

While the present invention has been described with reference to the exemplary embodiments described above, the present invention is not limited to the above-described exemplary embodiments. The form and details of the present invention can be changed within the scope of the present invention in various manners that can be understood by those skilled in the art.

### REFERENCE SIGNS LIST

100 model training apparatus
110 operation input unit
120 screen display unit
130 communication I/F unit
140 storage unit
141 body motion model
142 sensing data
143 body motion score information
144 vital model
145 program
150 arithmetic processing unit
151 sensing data acquisition unit
152 body motion score calculation unit
153 agitation determination unit
154 labeling unit
155 vital model training unit
156 output unit
157 attribute information acquisition unit
200 agitation determination system
300 sensor apparatus
310 body motion sensor
320 vital sensor
330 transmission/reception unit
400 bed terminal
410 transmission/reception unit
420 screen display unit
500 agitation determination apparatus
510 operation input unit
520 screen display unit
530 communication IF unit
540 storage unit
541 body motion model
542 vital model
543 sensing data
544 body motion score information
545 vital score information
546 result information
547 program
548 environmental information
550 arithmetic processing unit
551 sensing data acquisition unit
552 score calculation unit
553 agitation state determination unit
554 notification unit
555 labeling-purpose agitation determination unit
556 labeling unit
557 retraining unit
558 attribute information acquisition unit
600 supervisory data generation apparatus
601 CPU
602 ROM
603 RAM
604 program group
605 storage device
606 drive
607 communication interface
608 input/output interface
609 bus
610 recording medium
611 communication network
621 acquisition unit
622 calculation unit
623 determination unit
624 labeling unit

What is claimed is:

1. A supervisory data generation apparatus comprising:
at least one memory configured to store processing instructions; and
at least one processor configured to execute the processing instructions to: acquire body motion data according to a motion of a body of a target and vital data of the target;
calculate a body motion score serving as an index for determining an agitation state based on the acquired body motion data;
determine whether the target is agitated using the body motion score; and
label the vital data based on a result of the determination.

2. The supervisory data generation apparatus according to claim 1, wherein the at least one processor is configured to execute the processing instructions to
carry out the labeling with respect to a partial period in the vital data during which the corresponding body motion score satisfies a predetermined criterion.

3. The supervisory data generation apparatus according to claim 1, wherein the at least one processor is configured to execute the processing instructions to:
determine that the target is agitated with respect to a period during which the body motion score is equal to or higher than a first threshold value; and
carry out the labeling with respect to the period in the vital data during which the target is determined to be agitated.

4. The supervisory data generation apparatus according to claim 1, wherein the at least one processor is configured to execute the processing instructions to:
determine that the target is not agitated with respect to a period during which the body motion score is equal to or lower than a second threshold value; and
carry out the labeling with respect to the period in the vital data during which the target is determined not to be agitated.

5. The supervisory data generation apparatus according to claim 1, wherein the at least one processor is configured to execute the processing instructions to:
acquire attribute information indicating an attribute of the target; and
determine whether the target is agitated based on the acquired attribute information.

6. The supervisory data generation apparatus according to claim 1, wherein the at least one processor is configured to execute the processing instructions to:
acquire attribute information indicating an attribute of the target; and
determine whether to use the state information for training based on the acquired attribute information acquired.

7. A supervisory data generation method for causing a computer to:
acquire body motion data according to a motion of a body of a target and vital data of the target;
calculate a body motion score serving as an index for determining an agitation state based on the acquired body motion data;
determine whether the target is agitated based on the body motion score; and
label the vital data based on a result of the determination.

8.-11. (canceled)

12. An agitation determination apparatus comprising:
at least one memory configured to store processing instructions; and
at least one processor configured to execute the processing instructions to: acquire body motion data according to a motion of a body of a target and vital data according to a state of the body of the target;
calculate a body motion score serving as an index for determining an agitation state based on the acquired body motion data and also calculate a vital score serving as an index for determining the agitation state based on the acquired vital data;
determine to retrain a vital model used when the vital score is calculated based on the calculated body motion score and the vital score; and
retrain the vital model according to a result of the determination.

13. The agitation determination apparatus according to claim 12, wherein the at least one processor is configured to execute the processing instructions to
determine to carry out the retraining in a case where the body motion score satisfies a predetermined criterion and the vital score does not satisfy a predetermined criterion.

14. The agitation determination apparatus according to claim 12, wherein at least one processor is configured to execute the processing instructions to:
generate supervisory data by labeling the vital data based on the result of the determination; and
retrain the vital model by carrying out retraining using the generated supervisory data.

15. The agitation determination apparatus according to claim 14, wherein the at least one processor is configured to execute the processing instructions to
generate the supervisory data with respect to a part of the vital data.

16. The agitation determination apparatus according to claim 14, wherein the at least one processor is configured to execute the processing instructions to
generate the supervisory data with respect to a partial period in the vital data during which the corresponding body motion score satisfies a predetermined criterion.

17.-18. (canceled)
